SEQUENCE LISTING (171 sequences)

SEQ ID NO: 1 - DNA, 686 bp, Synthetic construct
  source 1..686: mol_type = other DNA
  misc_feature 1..686: note = 0-20 Heavy Chain VDJ/VJ
agctctgggagaagagccccagccccagaattcccaggagtttccattcggtgatcagcgctgaacacagaggactcaccatggagtttgggctgagctgggttttccttgttgctattataaaaggtgtccagtgtcaggtggagctcgtggagtcggggggaggcttggtcaagcctggggggtccctgagactctcctgttcagcctctggattcaagttcaatgactactatatgagttggatccggcaggctccaggaaaggggctggagtgggtttcatacattagtagttttggagttaccacatactacgccgactctgtcaagggccgcttcaccatctctatggacagcgacttgaagacactatatcttcaaatcaacagcctgagagtcgaggacacggccgtctacttctgtgcgagaggaaacaatttgtttgatgatagaggtctctttgaccactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 2 - DNA, 633 bp, Synthetic construct
  source 1..633: mol_type = other DNA
  misc_feature 1..633: note = 0-20 Light Chain VDJ/VJ
agtctgggcctaaggaagcagcactggtggtgcctcagccatggcctggaccgttctcctcctcggcctcctctctcactgcacaggctctgtgacctcctatgtgttgactcagccaccctcggtgtcagtggccccaggaaagacggccaggattacctgtgggggagacaacattggaagtaagagtgtgcactggtaccagcagaagccaggccaggcccctgaactggtcgtctatgacaataccgaccgggccgcagatgtccctgagcgattctctggctccaactctggcaacacggccaccctgaccatcatcagggtcgaagccggggatgaggccgactattactgtcaggtgcgggatacaggtacttttcagcatgtcttcggaagtgggaccaaggtcaccgtcctgggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagcta

SEQ ID NO: 3 - DNA, 681 bp, Synthetic construct
  source 1..681: mol_type = other DNA
  misc_feature 1..681: note = 1-2 Heavy Chain VDJ/VJ
agcactagaagccggcggtgtttccattcggtgatcagcactgaacacagaggactcaccatggagtttgggctgagctgggttttcctcgttgctcttttaagagctgtccagtgtcaggtgcagctggtggagtctgggggaggcgtggtccagcctgggaggtccctgagactctcctgtgcagcctctggattcagcttcagtaactttggcttccactgggtccgccacgttccaggcaaggggctggagtgggtggcacttatatcacatgatggaagtaataaggcatatgttggaggtcagagacactatgcagactccgtgaagggccgattcaccgtctccagagacaatgccaagaacagggtgtatctgcaattgaacagcctgaaaactgaggacacggctgtgtattattgtgcccgcgctgcttatgattctctgacgtactttgaattctggggccagggagccctggtcaccgtctcctcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 4 - DNA, 633 bp, Synthetic construct
  source 1..633: mol_type = other DNA
  misc_feature 1..633: note = 1-2 Light Chain VDJ/VJ
agtctgggcctaaggaagcagcactggtggtgcctcagccatggcctggaccgttctcctcctcggcctcctctctcactgcacagggtctgtgacctcctatgtgctgactcagccaccctcagtgtcagtggccccaggaaagacggccagccttacctgtgggggaaacaacattggaggaaaaggtgtgcactggtaccagcggaggccaggccaggcccctgtgctggtcgtctatcttgatagagaccggccctcaggggtccctgagcgattctctggctccaactctgggaacacggccaccctgaccatcagcagggtcgaagccggggatgaggccgactattactgtcaggtgtgggatagtaccagtgatcattgggtgttcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 5 - DNA, 649 bp, Synthetic construct
  source 1..649: mol_type = other DNA
  misc_feature 1..649: note = 5-1 Heavy Chain VDJ/VJ
tggggacccaaaaaccacacccctccttgggagaatcccctagatcacagctcctcaccatggactggacctggagcatccttttcttggtggcagcagcaacaggtggccactcccaggaatcccagggtcagctggtgcagtctggacctgaggtgaagaaacctggggccacagtgaaggtctcctgccgggcttctgcttacccctttggcaactatggtatcacctgggtgcgacaggtccctggacaaggacttgagtgggtgggatggatcagcgcgtacaccggtcacacaaagtttccacagaatttccagggcagagtcaccctgaccgcagacacatccacgagcacaggctacatggaactgaggagcctgacctctgacgacacggccgtctactactgtgcgagggggccgtgttgtagtagtccccggccctacgatatctggggccaagggacaatggtcaccgtctcttcagcatccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttccc

SEQ ID NO: 6 - DNA, 551 bp, Synthetic construct
  source 1..551: mol_type = other DNA
  misc_feature 1..551: note = 5-1 Light Chain VDJ/VJ
aggagtcagacccagtcaggacacagcatggacatgagggtccccgctcagctcctggggctcctgctgctctggctcccaggtgccgactgtgacatccagatgacccagtctccttccaccctgtctgcatctgtaggagacagagtcaccatcacttgccgggccagtcagagtattgatgactggttggcctggtatcagcacagtccagggaaagccccgaaactcctcatctatagggcgtctcgtttagaaagtggggtcccatcaaggttcagcggcagtggatctgggacagaattcactctcaccatcagcagcctgcagcctgatgattttgcaagttattactgccaacagtgttatacttattctcaaaccttcggccaagggaccagggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 7 - DNA, 686 bp, Synthetic construct
  source 1..686: mol_type = other DNA
  misc_feature 1..686: note = 2-6 Heavy Chain VDJ/VJ
agctctgggagaagagccccagccccagaattcccaggagtttccattcggtgatcagcactgaacacagaggactcaccatggagtttgggctgagctgggttttccttgttgctattataaaaggtgtccagtgtcaggtacagttggtggagtctgggggaggcttggtcaagcctggagggtctctgagactctcctgtgcagcctctggattcaccctcagtggctactacatgagctggatccgccaggctccagagaagggactggagtggatttcagatgttagtgggagtagttcttacacaaactacgcagactctgtgaagggccgattcaccatctccagagacaaccccaagaactcactgtatctgcaaatgaacagcctgagagccgaggacacggctatatattactgcgcgagaatttcttatacttctactggtcctttttactttgactcctggggccagggaatcctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgccctgaccagcggcgtgcacaccttcccagctgtcctacagtcctcagga

SEQ ID NO: 8 - DNA, 653 bp, Synthetic construct
  source 1..653: mol_type = other DNA
  misc_feature 1..653: note = 2-6 Light Chain VDJ/VJ
gctctgcttcagctgtgggcacaagaggcagcactcaggacaatctccagcatggcctggtctcctctcctcctcactctcctcgctcactgcacagggtcctgggcccagtctgtgctgacgcagccgccctcagtgtctgaggccccagggcagagggtcaccatctcctgcactgggagcagctccaacatcggggcaggttatgatgtccactggtaccagcagcttccaggaacagcccccaaactcctcatctatggtaacaacaatcggccctcagggatccctgaccgattctctggctccaagtctggcacctcagccgccctggccatcactgggctccaggctgaggatgaggctgattactactgccagtcctatgacagaagtctgagtggttatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagcta

SEQ ID NO: 9 - DNA, 679 bp, Synthetic construct
  source 1..679: mol_type = other DNA
  misc_feature 1..679: note = 9-1 Heavy Chain VDJ/VJ
agctctgagagaggagccttagccctggattccaaggcctatccacttggtgatcagcactgagcaccgaggattcaccatggaactggggctccgctgggttttccttgttgctattttagaaggtgtccagtgtgaggtgcagttggtggagtctgggggaggcctggtcaagccgggggggtccctgagactctcctgtgcagcctctggattcagcatcacgagttttagtttgcactgggtccgccagagtccagggaaggggctggagtgggtctcatccattagtgctagtagcagttacatagaatacgcagactcagcgaaggggcgcttcaccatctccagagacaacggcaagaactcgctctttctgcaattgaacagcctgagagtcgaggacacggctctttatttctgtgcgagagatagcgggcagcagctggatccgtttgactactggggccagggaaccctggtcacagtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 10 - DNA, 653 bp, Synthetic construct
  source 1..653: mol_type = other DNA
  misc_feature 1..653: note = 9-1 Light Chain VDJ/VJ
gctctgcttcagctgtgggcacaagaggcagcactcaggacaatctccagcatggcctggtctcctctcctcctcactctcctcgctcactgcacagggtcctgggcccagtctgtgctgacgcagccgccgtcagtgtctggggccccagggcagagggtcaccatctcctgcactgggaccagctccaacatcggggcaggttatgatgtacactggtaccaacagtttccaggaacggcccccaaactcctcatctatggaaacaacaatcggccctcaggggtccctgaccgattttctggctctaagtctgccacctcagcctccctggccatcactgggctccaggctgcggatgaggctgattattactgtcagtcctatgacaaacgcctctttggttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 11 - protein, 122 aa, Synthetic construct
  REGION 1..122: note = 0-20 Heavy Chain VDJ/VJ
QVELVESGGGLVKPGGSLRLSCSASGFKFNDYYMSWIRQAPGKGLEWVSYISSFGVTTYYADSVKGRFTISMDSDLKTLYLQINSLRVEDTAVYFCARGNNLFDDRGLFDHWGQGTLVTVSS

SEQ ID NO: 12 - protein, 108 aa, Synthetic construct
  REGION 1..108: note = 0-20 Light Chain VDJ/VJ
SYVLTQPPSVSVAPGKTARITCGGDNIGSKSVHWYQQKPGQAPELVVYDNTDRAADVPERFSGSNSGNTATLTIIRVEAGDEADYYCQVRDTGTFQHVFGSGTKVTVL

SEQ ID NO: 13 - protein, 127 aa, Synthetic construct
  REGION 1..127: note = 1-2 Heavy Chain VDJ/VJ
QVQLVESGGGVVQPGRSLRLSCAASGFSFSNFGFHWVRHVPGKGLEWVALISHDGSNKAYVGGQRHYADSVKGRFTVSRDNAKNRVYLQLNSLKTEDTAVYYCARAAYDSLTYFEFWGQGALVTVSS

SEQ ID NO: 14 - protein, 108 aa, Synthetic construct
  REGION 1..108: note = 1-2 Light Chain VDJ/VJ
SYVLTQPPSVSVAPGKTASLTCGGNNIGGKGVHWYQRRPGQAPVLVVYLDRDRPSGVPERFSGSNSGNTATLTISRVEAGDEADYYCQVWDSTSDHWVFGGGTKLTVL

SEQ ID NO: 15 - protein, 121 aa, Synthetic construct
  REGION 1..121: note = 5-1 Heavy Chain VDJ/VJ
QGQLVQSGPEVKKPGATVKVSCRASAYPFGNYGITWVRQVPGQGLEWVGWISAYTGHTKFPQNFQGRVTLTADTSTSTGYMELRSLTSDDTAVYYCARGPCCSSPRPYDIWGQGTMVTVSS

SEQ ID NO: 16 - protein, 107 aa, Synthetic construct
  REGION 1..107: note = 5-1 Light Chain VDJ/VJ
DIQMTQSPSTLSASVGDRVTITCRASQSIDDWLAWYQHSPGKAPKLLIYRASRLESGVPSRFSGSGSGTEFTLTISSLQPDDFASYYCQQCYTYSQTFGQGTRVEIK

SEQ ID NO: 17 - protein, 122 aa, Synthetic construct
  REGION 1..122: note = 2-6 Heavy Chain VDJ/VJ
QVQLVESGGGLVKPGGSLRLSCAASGFTLSGYYMSWIRQAPEKGLEWISDVSGSSSYTNYADSVKGRFTISRDNPKNSLYLQMNSLRAEDTAIYYCARISYTSTGPFYFDSWGQGILVTVSS

SEQ ID NO: 18 - protein, 111 aa, Synthetic construct
  REGION 1..111: note = 2-6 Light Chain VDJ/VJ
QSVLTQPPSVSEAPGQRVTISCTGSSSNIGAGYDVHWYQQLPGTAPKLLIYGNNNRPSGIPDRFSGSKSGTSAALAITGLQAEDEADYYCQSYDRSLSGYVFGTGTKVTVL

SEQ ID NO: 19 - protein, 120 aa, Synthetic construct
  REGION 1..120: note = 9-1 Heavy Chain VDJ/VJ
EVQLVESGGGLVKPGGSLRLSCAASGFSITSFSLHWVRQSPGKGLEWVSSISASSSYIEYADSAKGRFTISRDNGKNSLFLQLNSLRVEDTALYFCARDSGQQLDPFDYWGQGTLVTVSS

SEQ ID NO: 20 - protein, 111 aa, Synthetic construct
  REGION 1..111: note = 9-1 Light Chain VDJ/VJ
QSVLTQPPSVSGAPGQRVTISCTGTSSNIGAGYDVHWYQQFPGTAPKLLIYGNNNRPSGVPDRFSGSKSATSASLAITGLQAADEADYYCQSYDKRLFGWVFGGGTKLTVL

SEQ ID NO: 21 - DNA, 24 bp, Synthetic construct
  source 1..24: mol_type = other DNA
  misc_feature 1..24: note = 0-20 Heavy Chain CDR1
ggattcaagttcaatgactactat

SEQ ID NO: 22 - DNA, 18 bp, Synthetic construct
  source 1..18: mol_type = other DNA
  misc_feature 1..18: note = 0-20 Light Chain CDR1
aacattggaagtaagagt

SEQ ID NO: 23 - DNA, 24 bp, Synthetic construct
  source 1..24: mol_type = other DNA
  misc_feature 1..24: note = 1-2 Heavy Chain CDR1
ggattcagcttcagtaactttggc

SEQ ID NO: 24 - DNA, 18 bp, Synthetic construct
  source 1..18: mol_type = other DNA
  misc_feature 1..18: note = 1-2 Light Chain CDR1
aacattggaggaaaaggt

SEQ ID NO: 25 - DNA, 24 bp, Synthetic construct
  source 1..24: mol_type = other DNA
  misc_feature 1..24: note = 5-1 Heavy Chain CDR1
gcttacccctttggcaactatggt

SEQ ID NO: 26 - DNA, 18 bp, Synthetic construct
  source 1..18: mol_type = other DNA
  misc_feature 1..18: note = 5-1 Light Chain CDR1
cagagtattgatgactgg

SEQ ID NO: 27 - DNA, 24 bp, Synthetic construct
  source 1..24: mol_type = other DNA
  misc_feature 1..24: note = 2-6 Heavy Chain CDR1
ggattcaccctcagtggctactac

SEQ ID NO: 28 - DNA, 27 bp, Synthetic construct
  source 1..27: mol_type = other DNA
  misc_feature 1..27: note = 2-6 Light Chain CDR1; 9-1 Light Chain CDR1
agctccaacatcggggcaggttatgat

SEQ ID NO: 29 - DNA, 24 bp, Synthetic construct
  source 1..24: mol_type = other DNA
  misc_feature 1..24: note = 9-1 Heavy Chain CDR1
ggattcagcatcacgagttttagt

SEQ ID NO: 30 - DNA, 24 bp, Synthetic construct
  source 1..24: mol_type = other DNA
  misc_feature 1..24: note = 0-20 Heavy Chain CDR2
attagtagttttggagttaccaca

SEQ ID NO: 31 - DNA, 24 bp, Synthetic construct
  source 1..24: mol_type = other DNA
  misc_feature 1..24: note = 1-2 Heavy Chain CDR2
atatcacatgatggaagtaataag

SEQ ID NO: 32 - DNA, 24 bp, Synthetic construct
  source 1..24: mol_type = other DNA
  misc_feature 1..24: note = 5-1 Heavy Chain CDR2
atcagcgcgtacaccggtcacaca

SEQ ID NO: 33 - DNA, 24 bp, Synthetic construct
  source 1..24: mol_type = other DNA
  misc_feature 1..24: note = 2-6 Heavy Chain CDR2
gttagtgggagtagttcttacaca

SEQ ID NO: 34 - DNA, 24 bp, Synthetic construct
  source 1..24: mol_type = other DNA
  misc_feature 1..24: note = 9-1 Heavy Chain CDR2
attagtgctagtagcagttacata

SEQ ID NO: 35 - DNA, 45 bp, Synthetic construct
  source 1..45: mol_type = other DNA
  misc_feature 1..45: note = 0-20 Heavy Chain CDR3
gcgagaggaaacaatttgtttgatgatagaggtctctttgaccac

SEQ ID NO: 36 - DNA, 33 bp, Synthetic construct
  source 1..33: mol_type = other DNA
  misc_feature 1..33: note = 0-20 Light Chain CDR3
caggtgcgggatacaggtacttttcagcatgtc

SEQ ID NO: 37 - DNA, 60 bp, Synthetic construct
  source 1..60: mol_type = other DNA
  misc_feature 1..60: note = 1-2 Heavy Chain CDR3
gacacggctgtgtattattgtgcccgcgctgcttatgattctctgacgtactttgaattc

SEQ ID NO: 38 - DNA, 33 bp, Synthetic construct
  source 1..33: mol_type = other DNA
  misc_feature 1..33: note = 1-2 Light Chain CDR3
caggtgtgggatagtaccagtgatcattgggtg

SEQ ID NO: 39 - DNA, 42 bp, Synthetic construct
  source 1..42: mol_type = other DNA
  misc_feature 1..42: note = 5-1 Heavy Chain CDR3
gcgagggggccgtgttgtagtagtccccggccctacgatatc

SEQ ID NO: 40 - DNA, 27 bp, Synthetic construct
  source 1..27: mol_type = other DNA
  misc_feature 1..27: note = 5-1 Light Chain CDR3
caacagtgttatacttattctcaaacc

SEQ ID NO: 41 - DNA, 45 bp, Synthetic construct
  source 1..45: mol_type = other DNA
  misc_feature 1..45: note = 2-6 Heavy Chain CDR3
gcgagaatttcttatacttctactggtcctttttactttgactcc

SEQ ID NO: 42 - DNA, 33 bp, Synthetic construct
  source 1..33: mol_type = other DNA
  misc_feature 1..33: note = 2-6 Light Chain CDR3
cagtcctatgacagaagtctgagtggttatgtc

SEQ ID NO: 43 - DNA, 39 bp, Synthetic construct
  source 1..39: mol_type = other DNA
  misc_feature 1..39: note = 9-1 Heavy Chain CDR3
gcgagagatagcgggcagcagctggatccgtttgactac

SEQ ID NO: 44 - DNA, 33 bp, Synthetic construct
  source 1..33: mol_type = other DNA
  misc_feature 1..33: note = 9-1 Light Chain CDR3
cagtcctatgacaaacgcctctttggttgggtg

SEQ ID NO: 45 - protein, 8 aa, Synthetic construct
  REGION 1..8: note = 0-20 Heavy Chain CDR1
GFKFNDYY

SEQ ID NO: 46 - protein, 6 aa, Synthetic construct
  REGION 1..6: note = 0-20 Light Chain CDR1
NIGSKS

SEQ ID NO: 47 - protein, 8 aa, Synthetic construct
  REGION 1..8: note = 1-2 Heavy Chain CDR1
GFSFSNFG

SEQ ID NO: 48 - protein, 6 aa, Synthetic construct
  REGION 1..6: note = 1-2 Light Chain CDR1
NIGGKG

SEQ ID NO: 49 - protein, 8 aa, Synthetic construct
  REGION 1..8: note = 5-1 Heavy Chain CDR1
AYPFGNYG

SEQ ID NO: 50 - protein, 6 aa, Synthetic construct
  REGION 1..6: note = 5-1 Light Chain CDR1
QSIDDW

SEQ ID NO: 51 - protein, 8 aa, Synthetic construct
  REGION 1..8: note = 2-6 Heavy Chain CDR1
GFTLSGYY

SEQ ID NO: 52 - protein, 9 aa, Synthetic construct
  REGION 1..9: note = 2-6 Light Chain CDR1; 9-1 Light Chain CDR1
SSNIGAGYD

SEQ ID NO: 53 - protein, 8 aa, Synthetic construct
  REGION 1..8: note = 9-1 Heavy Chain CDR1
GFSITSFS

SEQ ID NO: 54 - protein, 8 aa, Synthetic construct
  REGION 1..8: note = 0-20 Heavy Chain CDR2
ISSFGVTT

SEQ ID NO: 55 - protein, 8 aa, Synthetic construct
  REGION 1..8: note = 1-2 Heavy Chain CDR2
ISHDGSNK

SEQ ID NO: 56 - protein, 8 aa, Synthetic construct
  REGION 1..8: note = 5-1 Heavy Chain CDR2
ISAYTGHT

SEQ ID NO: 57 - protein, 8 aa, Synthetic construct
  REGION 1..8: note = 2-6 Heavy Chain CDR2
VSGSSSYT

SEQ ID NO: 58 - protein, 8 aa, Synthetic construct
  REGION 1..8: note = 9-1 Heavy Chain CDR2
ISASSSYI

SEQ ID NO: 59 - protein, 15 aa, Synthetic construct
  REGION 1..15: note = 0-20 Heavy Chain CDR3
ARGNNLFDDRGLFDH

SEQ ID NO: 60 - protein, 11 aa, Synthetic construct
  REGION 1..11: note = 0-20 Light Chain CDR3
QVRDTGTFQHV

SEQ ID NO: 61 - protein, 20 aa, Synthetic construct
  REGION 1..20: note = 1-2 Heavy Chain CDR3
DTAVYYCARAAYDSLTYFEF

SEQ ID NO: 62 - protein, 11 aa, Synthetic construct
  REGION 1..11: note = 1-2 Light Chain CDR3
QVWDSTSDHWV

SEQ ID NO: 63 - protein, 14 aa, Synthetic construct
  REGION 1..14: note = 5-1 Heavy Chain CDR3
ARGPCCSSPRPYDI

SEQ ID NO: 64 - protein, 9 aa, Synthetic construct
  REGION 1..9: note = 5-1 Light Chain CDR3
QQCYTYSQT

SEQ ID NO: 65 - protein, 15 aa, Synthetic construct
  REGION 1..15: note = 2-6 Heavy Chain CDR3
ARISYTSTGPFYFDS

SEQ ID NO: 66 - protein, 11 aa, Synthetic construct
  REGION 1..11: note = 2-6 Light Chain CDR3
QSYDRSLSGYV

SEQ ID NO: 67 - protein, 13 aa, Synthetic construct
  REGION 1..13: note = 9-1 Heavy Chain CDR3
ARDSGQQLDPFDY

SEQ ID NO: 68 - protein, 11 aa, Synthetic construct
  REGION 1..11: note = 9-1 Light Chain CDR3
QSYDKRLFGWV

SEQ ID NO: 69 - DNA, 700 bp, Synthetic construct
  source 1..700: mol_type = other DNA
  misc_feature 1..700: note = 1-33 Heavy Chain
aggtctcagagaggagccttagccctggactccaaggcctttccacttggtgatcagcactgagcacagaggactcaccatggaattggggctgagctgggttttccttgttgctattttagaaggtgtccagtgtgaagtgcagctggtggagtctgggggaaccttggtccagcctggggggtccctgagactctcctgtgcaatctctggattcaattttagtgattatcggatgaactgggtccgccaggctccagggaaggggctggagtgggtggccaacataaaggaagatggacgtgagaagtactatgtggcctctgtgaagggccggttcaccatctccagagacaacgccaagaaatcagtgtatctgcagatgaacagcctgagagtcgaggacacggctgtttattactgcgcgagaggggggcgtctacgtcattttggcaattattactattatcctggcatgggcgtctggggccgagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcagga

SEQ ID NO: 70 - DNA, 569 bp, Synthetic construct
  source 1..569: mol_type = other DNA
  misc_feature 1..569: note = 1-33 Light Chain
atgggggatcaggactcctcagttcaccttctcacaatgaggctccctgctcagctcctggggctgctaatgctctgggtctctggatccagtggggatattgtgatgactcagtctccactctccctgcccgtcacccctggagagccggcctccatctcctgcaggtctagtcagagcctcctccataataatacatacaattatttggattggtatgtgcagaaggcagggcagtctccacaactcctgatctatttggcttcttatcgggcctccggggtccctgacaggttcagtggcagcggatcaggcacagattttacactgaagatcaacagagtggaggctgtggatgttggggtttattactgcatgcaatctctacaaacccccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 71 - protein, 127 aa, Synthetic construct
  REGION 1..127: note = 1-33 Heavy Chain
EVQLVESGGTLVQPGGSLRLSCAISGFNFSDYRMNWVRQAPGKGLEWVANIKEDGREKYYVASVKGRFTISRDNAKKSVYLQMNSLRVEDTAVYYCARGGRLRHFGNYYYYPGMGVWGRGTTVTVSS

SEQ ID NO: 72 - protein, 112 aa, Synthetic construct
  REGION 1..112: note = 1-33 Light Chain
DIVMTQSPLSLPVTPGEPASISCRSSQSLLHNNTYNYLDWYVQKAGQSPQLLIYLASYRASGVPDRFSGSGSGTDFTLKINRVEAVDVGVYYCMQSLQTPLTFGGGTKVEIK

SEQ ID NO: 73 - DNA, 24 bp, Synthetic construct
  source 1..24: mol_type = other DNA
  misc_feature 1..24: note = 1-33 Heavy Chain CDR1
ggattcaattttagtgattatcgg

SEQ ID NO: 74 - DNA, 24 bp, Synthetic construct
  source 1..24: mol_type = other DNA
  misc_feature 1..24: note = 1-33 Heavy Chain CDR2
ataaaggaagatggacgtgagaag

SEQ ID NO: 75 - DNA, 60 bp, Synthetic construct
  source 1..60: mol_type = other DNA
  misc_feature 1..60: note = 1-33 Heavy Chain CDR3
gcgagaggggggcgtctacgtcattttggcaattattactattatcctggcatgggcgtc

SEQ ID NO: 76 - DNA, 33 bp, Synthetic construct
  source 1..33: mol_type = other DNA
  misc_feature 1..33: note = 1-33 Light Chain CDR1
cagagcctcctccataataatacatacaattat

SEQ ID NO: 77 - skipped

SEQ ID NO: 78 - DNA, 27 bp, Synthetic construct
  source 1..27: mol_type = other DNA
  misc_feature 1..27: note = 1-33 Light Chain CDR3
atgcaatctctacaaacccccctcact

SEQ ID NO: 79 - protein, 8 aa, Synthetic construct
  REGION 1..8: note = 1-33 Heavy Chain CDR1
GFNFSDYR

SEQ ID NO: 80 - protein, 8 aa, Synthetic construct
  REGION 1..8: note = 1-33 Heavy Chain CDR2
IKEDGREK

SEQ ID NO: 81 - protein, 20 aa, Synthetic construct
  REGION 1..20: note = 1-33 Heavy Chain CDR3
ARGGRLRHFGNYYYYPGMGV

SEQ ID NO: 82 - protein, 11 aa, Synthetic construct
  REGION 1..11: note = 1-33 Light Chain CDR1
QSLLHNNTYNY

SEQ ID NO: 83 - skipped

SEQ ID NO: 84 - protein, 9 aa, Synthetic construct
  REGION 1..9: note = 1-33 Light Chain CDR3
MQSLQTPLT

SEQ ID NO: 85 - DNA, 679 bp, Synthetic construct
  source 1..679: mol_type = other DNA
  misc_feature 1..679: note = 1-45 Heavy Chain
tctgagagaggagccttcgccctggattccaaggcctatccacttggtgatcagcactgagcaccgaggattcaccatggatctggggctccgctgggttttccttattactattttagaaggtgtccactgtgacgaacaactggtggagtctgggggacgcctggtcaagcctggggggtccctgagactctcctgtgcagcctcgggattcaccatcagtggctataacttctgctgggtccgccaggctccgggggaggggctgcagtggctcgcttccataactggtcttggtaattacatctcctatgccgactcactgcgaggccgatccactgtctccagagacaatgccaagaagttactttatctacaactgaacagcctgcgacccgaagacacggctgtatattactgtgcgagagtccatctggggcggcagacacacggcttcgatctctggggccaagggacaatggtcaccgtcgcttcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 86 - DNA, 653 bp, Synthetic construct
  source 1..653: mol_type = other DNA
  misc_feature 1..653: note = 1-45 Light Chain
gctctgcttcagctgtgggcacaagaggcagcactcaggacaatctccagcatggcctggtctcctctcctcctcactctcctcgctcactgcacagggtcctgggcccagtctgtgctgacgcagccgccctcagtgtctggggccccagggcagacggtcaccatctcctgctctgggagcgactccaacatcggggcaggttatgaagtacactggtaccagcaacttccaggaacagcccccaagttcctcatctttggctacaacaatcggccctcaggaatccctgaccgattctctggctccgcgtctggcacctcggcctccctggccatcactgggctccaggctgaagatgaggctgtttattactgccagtcctttgacaggagcctggatggatatgccttcggaactgggacccaggtcaccgtcctaagtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagcta

SEQ ID NO: 87 - protein, 121 aa, Synthetic construct
  REGION 1..121: note = 1-45 Heavy Chain
DEQLVESGGRLVKPGGSLRLSCAASGFTISGYNFCWVRQAPGEGLQWLASITGLGNYISYADSLRGRSTVSRDNAKKLLYLQLNSLRPEDTAVYYCARVHLGRQTHGFDLWGQGTMVTVAS

SEQ ID NO: 88 - protein, 111 aa, Synthetic construct
  REGION 1..111: note = 1-45 Light Chain
QSVLTQPPSVSGAPGQTVTISCSGSDSNIGAGYEVHWYQQLPGTAPKFLIFGYNNRPSGIPDRFSGSASGTSASLAITGLQAEDEAVYYCQSFDRSLDGYAFGTGTQVTVL

SEQ ID NO: 89 - DNA, 24 bp, Synthetic construct
  source 1..24: mol_type = other DNA
  misc_feature 1..24: note = 1-45 Heavy Chain CDR1
ggattcaccatcagtggctataac

SEQ ID NO: 90 - DNA, 24 bp, Synthetic construct
  source 1..24: mol_type = other DNA
  misc_feature 1..24: note = 1-45 Heavy Chain CDR2
ataactggtcttggtaattacatc

SEQ ID NO: 91 - DNA, 42 bp, Synthetic construct
  source 1..42: mol_type = other DNA
  misc_feature 1..42: note = 1-45 Heavy Chain CDR3
gcgagagtccatctggggcggcagacacacggcttcgatctc

SEQ ID NO: 92 - DNA, 27 bp, Synthetic construct
  source 1..27: mol_type = other DNA
  misc_feature 1..27: note = 1-45 Light Chain CDR1
gactccaacatcggggcaggttatgaa

SEQ ID NO: 93 - skipped

SEQ ID NO: 94 - DNA, 33 bp, Synthetic construct
  source 1..33: mol_type = other DNA
  misc_feature 1..33: note = 1-45 Light Chain CDR3
cagtcctttgacaggagcctggatggatatgcc

SEQ ID NO: 95 - protein, 8 aa, Synthetic construct
  REGION 1..8: note = 1-45 Heavy Chain CDR1
GFTISGYN

SEQ ID NO: 96 - protein, 8 aa, Synthetic construct
  REGION 1..8: note = 1-45 Heavy Chain CDR2
ITGLGNYI

SEQ ID NO: 97 - protein, 14 aa, Synthetic construct
  REGION 1..14: note = 1-45 Heavy Chain CDR3
ARVHLGRQTHGFDL

SEQ ID NO: 98 - protein, 9 aa, Synthetic construct
  REGION 1..9: note = 1-45 Light Chain CDR1
DSNIGAGYE

SEQ ID NO: 99 - skipped

SEQ ID NO: 100 - protein, 11 aa, Synthetic construct
  REGION 1..11: note = 1-45 Light Chain CDR3
QSFDRSLDGYA

SEQ ID NO: 101 - DNA, 682 bp, Synthetic construct
  source 1..682: mol_type = other DNA
  misc_feature 1..682: note = 1-47 Heavy Chain
agctctgagagaggagccttagccctggattccaaggcctatccacttggtgatcagcactgagcaccgaagattcaccatggaactggggctccgctgggttttccttcttgctattttagaaggtgtccagtgtgaggtgcagttggtggagtctgggggaggcctggtcaagcctggggggtccctgagactctcctgtgcagcctctggattcaccttcagtggctataacatggtctgggtccgccaggctccagggagggggctggagtgggtctcatccattactagtggttctaattacatagagtatgcagactcagtgaagggccgattcaccatctccagagacaacgccaagaactcactgtttctgcaaatgaacagcctgagagccgaggacacggctctctattactgtgcgagagtccatttggggcgacaaacatatggttttgatatctggggccaaggaacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcagga

SEQ ID NO: 102 - DNA, 653 bp, Synthetic construct
  source 1..653: mol_type = other DNA
  misc_feature 1..653: note = 1-47 Light Chain
gctctgcttcagctgtgggcacaagaggcagcactcaggacaatctccagcatggcctggtctcctctcctcctcactctcctcgctcactgcacagggtcctgggcccagtctgtgctgacgcagccgccctcagtgtctggggccccagggcagagggtcaccatctcctgcactgggagcagctccaacatcggggcaggttacgatgtacactggtaccagcaacttccaggaacagcccccaaattcctcatctatggtaatattaatcgaccctcaggggtccctgaccgattctctggctccaactctggcacctcagcctccctggccatcactgggctccagccagaggatgaggctgactattactgccactcctatgaccgcagcctgggtggttatgtcttcggaactgggaccaaagtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagcta

SEQ ID NO: 103 - protein, 121 aa, Synthetic construct
  REGION 1..121: note = 1-47 Heavy Chain
EVQLVESGGGLVKPGGSLRLSCAASGFTFSGYNMVWVRQAPGRGLEWVSSITSGSNYIEYADSVKGRFTISRDNAKNSLFLQMNSLRAEDTALYYCARVHLGRQTYGFDIWGQGTMVTVSS

SEQ ID NO: 104 - protein, 111 aa, Synthetic construct
  REGION 1..111: note = 1-47 Light Chain
QSVLTQPPSVSGAPGQRVTISCTGSSSNIGAGYDVHWYQQLPGTAPKFLIYGNINRPSGVPDRFSGSNSGTSASLAITGLQPEDEADYYCHSYDRSLGGYVFGTGTKVTVL

SEQ ID NO: 105 - DNA, 24 bp, Synthetic construct
  source 1..24: mol_type = other DNA
  misc_feature 1..24: note = 1-47 Heavy Chain CDR1
ggattcaccttcagtggctataac

SEQ ID NO: 106 - DNA, 24 bp, Synthetic construct
  source 1..24: mol_type = other DNA
  misc_feature 1..24: note = 1-47 Heavy Chain CDR2
attactagtggttctaattacata

SEQ ID NO: 107 - DNA, 42 bp, Synthetic construct
  source 1..42: mol_type = other DNA
  misc_feature 1..42: note = 1-47 Heavy Chain CDR3
gcgagagtccatttggggcgacaaacatatggttttgatatc

SEQ ID NO: 108 - DNA, 27 bp, Synthetic construct
  source 1..27: mol_type = other DNA
  misc_feature 1..27: note = 1-47 Light Chain CDR1
agctccaacatcggggcaggttacgat

SEQ ID NO: 109 - skipped

SEQ ID NO: 110 - DNA, 33 bp, Synthetic construct
  source 1..33: mol_type = other DNA
  misc_feature 1..33: note = 1-47 Light Chain CDR3
cactcctatgaccgcagcctgggtggttatgtc

SEQ ID NO: 111 - protein, 8 aa, Synthetic construct
  REGION 1..8: note = 1-47 Heavy Chain CDR1
GFTFSGYN

SEQ ID NO: 112 - protein, 8 aa, Synthetic construct
  REGION 1..8: note = 1-47 Heavy Chain CDR2
ITSGSNYI

SEQ ID NO: 113 - protein, 14 aa, Synthetic construct
  REGION 1..14: note = 1-47 Heavy Chain CDR3
ARVHLGRQTYGFDI

SEQ ID NO: 114 - protein, 9 aa, Synthetic construct
  REGION 1..9: note = 1-47 Light Chain CDR1
SSNIGAGYD

SEQ ID NO: 115 - skipped

SEQ ID NO: 116 - protein, 11 aa, Synthetic construct
  REGION 1..11: note = 1-47 Light Chain CDR3
HSYDRSLGGYV

SEQ ID NO: 117 - DNA, 685 bp, Synthetic construct
  source 1..685: mol_type = other DNA
  misc_feature 1..685: note = 1-48 Heavy Chain
agctctgagagaggagcccagccctgggattttcaggtgttttcatttggtgatcaggactgaacagagagaactcaccatggagtttgggctgaggtggctttttcttgtggctattttaaaaggtgtccagtgtgaggtgcaactattggactctgggggaggcttggtacagccgggggggtccctgagactgtcctgtgcagcctctggattcacctttagcaactatgccatgagctgggtccgccaggctcccgggaaggggctggagtgggtcgcacgtgttggtgcaagtggttatcccacatattatgcagactccgtgaagggccggttcaccatctccagagacaattccgaggacacactacatctgcaattgaagaccctgagagtcgaagacacggccctttattattgtgcgagagatggattcggggatagtagtggccaagaacttgactactggggccagggaaccttggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 118 - DNA, 566 bp, Synthetic construct
  source 1..566: mol_type = other DNA
  misc_feature 1..566: note = 1-48 Light Chain
gggactgatcaggactcctcagttcaccttctcacaatgaggctccctgctcagctcctggggctgctaatgctctgggtctctggatccagtggggatattgtgatgactcagtctccactctccctgcccgtcacccctggagagccggcctccatctcgtgtaggtctagtcagagtctcctgcagagtaatggatacaactatttaaattggtacctgaagaagccagggcagtctccacagctcttgatctatttgggtttcatgcgggcctccggggtccctgacaggttcaagggcagtggatcaggcacagattttacactgaaaatcagcagagtggaggctgaggatgttggggtttattactgcatgcaagctctacaaactcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 119 - protein, 122 aa, Synthetic construct
  REGION 1..122: note = 1-48 Heavy Chain
EVQLLDSGGGLVQPGGSLRLSCAASGFTFSNYAMSWVRQAPGKGLEWVARVGASGYPTYYADSVKGRFTISRDNSEDTLHLQLKTLRVEDTALYYCARDGFGDSSGQELDYWGQGTLVTVSS

SEQ ID NO: 120 - protein, 111 aa, Synthetic construct
  REGION 1..111: note = 1-48 Light Chain
DIVMTQSPLSLPVTPGEPASISCRSSQSLLQSNGYNYLNWYLKKPGQSPQLLIYLGFMRASGVPDRFKGSGSGTDFTLKISRVEAEDVGVYYCMQALQTRTFGQGTKVEIK

SEQ ID NO: 121 - DNA, 24 bp, Synthetic construct
  source 1..24: mol_type = other DNA
  misc_feature 1..24: note = 1-48 Heavy Chain CDR1
ggattcacctttagcaactatgcc

SEQ ID NO: 122 - DNA, 24 bp, Synthetic construct
  source 1..24: mol_type = other DNA
  misc_feature 1..24: note = 1-48 Heavy Chain CDR2
gttggtgcaagtggttatcccaca

SEQ ID NO: 123 - DNA, 45 bp, Synthetic construct
  source 1..45: mol_type = other DNA
  misc_feature 1..45: note = 1-48 Heavy Chain CDR3
gcgagagatggattcggggatagtagtggccaagaacttgactac

SEQ ID NO: 124 - DNA, 33 bp, Synthetic construct
  source 1..33: mol_type = other DNA
  misc_feature 1..33: note = 1-48 Light Chain CDR1
cagagtctcctgcagagtaatggatacaactat

SEQ ID NO: 125 - skipped

SEQ ID NO: 126 - DNA, 24 bp, Synthetic construct
  source 1..24: mol_type = other DNA
  misc_feature 1..24: note = 1-48 Light Chain CDR3
atgcaagctctacaaactcggacg

SEQ ID NO: 127 - protein, 8 aa, Synthetic construct
  REGION 1..8: note = 1-48 Heavy Chain CDR1
GFTFSNYA

SEQ ID NO: 128 - protein, 8 aa, Synthetic construct
  REGION 1..8: note = 1-48 Heavy Chain CDR2
VGASGYPT

SEQ ID NO: 129 - protein, 15 aa, Synthetic construct
  REGION 1..15: note = 1-48 Heavy Chain CDR3
ARDGFGDSSGQELDY

SEQ ID NO: 130 - protein, 11 aa, Synthetic construct
  REGION 1..11: note = 1-48 Light Chain CDR1
QSLLQSNGYNY

SEQ ID NO: 131 - skipped

SEQ ID NO: 132 - protein, 8 aa, Synthetic construct
  REGION 1..8: note = 1-48 Light Chain CDR3
MQALQTRT

SEQ ID NO: 133 - DNA, 688 bp, Synthetic construct
  source 1..688: mol_type = other DNA
  misc_feature 1..688: note = 2-1 Heavy Chain
gagagaggagccttagccctggattccaaggcctctccacttggtgatcagcactgagcaccgaggattcaccatggaactggggctccgctgggttttccttgttgctattttagaaggtttccaatgtgaagtgcagttggtggagtctgggggaggcctggtcaagcctggggggtccctgagactctcttgtacagcgtctggattcagacttagtagttatggcatgaattgggtccgccaggcaccagggaagggactggagtgggtctcgtccatcagtggcgggagtaattacatagactatgcagacctactgaagggccggttcaccatctccagagacaacgccaagaattcactgtatctgcaaatgaacagcctgagagccgaggacacggctgtatattactgtgtgagaagtctctatgattcgggacattattataatcccaaatggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 134 - DNA, 680 bp, Synthetic construct
  source 1..680: mol_type = other DNA
  misc_feature 1..680: note = 2-1 Light Chain
cctcgctcactgcacaggtgactggatacaggtccaggggaggggccctgggaagcctatggaatcttgctttctcctgttgtctctagaaggcgaatactgatgcctgtgtctctcccattcccagggtcctgggcccagactgcactgacgcagccgccctcagtgtctggggccccagggcagagggtcatcatctcctgcactgggaccacctccaacatcggggcaggttatgatgtacattggtaccagcagcttccaggaacagcccccaagaccctcatctatggtaacatcaatcggccctcaggagtccctgaccgattctttggctccaagtctggcacctcagcctccctggtcatcactgggctccaggctgaggatgaggctgattattactgccactcctatgacagaaccctgagagaagtgttcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 135 - protein, 125 aa, Synthetic construct
  REGION 1..125: note = 2-1 Heavy Chain
EVQLVESGGGLVKPGGSLRLSCTASGFRLSSYGMNWVRQAPGKGLEWVSSISGGSNYIDYADLLKGRFTISRDNAKNSLYLQMNSLRAEDTAVYYCVRSLYDSGHYYNPKWFDPWGQGTLVTVSS

SEQ ID NO: 136 - protein, 110 aa, Synthetic construct
  REGION 1..110: note = 2-1 Light Chain
QTALTQPPSVSGAPGQRVIISCTGTTSNIGAGYDVHWYQQLPGTAPKTLIYGNINRPSGVPDRFFGSKSGTSASLVITGLQAEDEADYYCHSYDRTLREVFGGGTKLTVL

SEQ ID NO: 137 - DNA, 24 bp, Synthetic construct
  source 1..24: mol_type = other DNA
  misc_feature 1..24: note = 2-1 Heavy Chain CDR1
ggattcagacttagtagttatggc

SEQ ID NO: 138 - DNA, 24 bp, Synthetic construct
  source 1..24: mol_type = other DNA
  misc_feature 1..24: note = 2-1 Heavy Chain CDR2
atcagtggcgggagtaattacata

SEQ ID NO: 139 - DNA, 54 bp, Synthetic construct
  source 1..54: mol_type = other DNA
  misc_feature 1..54: note = 2-1 Heavy Chain CDR3
gtgagaagtctctatgattcgggacattattataatcccaaatggttcgacccc

SEQ ID NO: 140 - DNA, 27 bp, Synthetic construct
  source 1..27: mol_type = other DNA
  misc_feature 1..27: note = 2-1 Light Chain CDR1
acctccaacatcggggcaggttatgat

SEQ ID NO: 141 - skipped

SEQ ID NO: 142 - DNA, 30 bp, Synthetic construct
  source 1..30: mol_type = other DNA
  misc_feature 1..30: note = 2-1 Light Chain CDR3
cactcctatgacagaaccctgagagaagtg

SEQ ID NO: 143 - protein, 8 aa, Synthetic construct
  REGION 1..8: note = 2-1 Heavy Chain CDR1
GFRLSSYG

SEQ ID NO: 144 - protein, 8 aa, Synthetic construct
  REGION 1..8: note = 2-1 Heavy Chain CDR2
ISGGSNYI

SEQ ID NO: 145 - protein, 18 aa, Synthetic construct
  REGION 1..18: note = 2-1 Heavy Chain CDR3
VRSLYDSGHYYNPKWFDP

SEQ ID NO: 146 - protein, 9 aa, Synthetic construct
  REGION 1..9: note = 2-1 Light Chain CDR1
TSNIGAGYD

SEQ ID NO: 147 - protein, 10 aa, Synthetic construct
  REGION 1..10: note = 2-1 Light Chain CDR3
HSYDRTLREV

SEQ ID NO: 148 - protein, 22 aa, Synthetic construct
  REGION 1..22: note = 1-33 Heavy Chain CDR3
CARGGRLRHFGNYYYYPGMGVW

SEQ ID NO: 149 - protein, 16 aa, Synthetic construct
  REGION 1..16: note = 1-45 Heavy Chain CDR3
CARVHLGRQTHGFDLW

SEQ ID NO: 150 - protein, 16 aa, Synthetic construct
  REGION 1..16: note = 1-47 Heavy Chain CDR3
CARVHLGRQTYGFDIW

SEQ ID NO: 151 - protein, 17 aa, Synthetic construct
  REGION 1..17: note = 1-48 Heavy Chain CDR3
CARDGFGDSSGQELDYW

SEQ ID NO: 152 - protein, 20 aa, Synthetic construct
  REGION 1..20: note = 2-1 Heavy Chain CDR3
CVRSLYDSGHYYNPKWFDPW

SEQ ID NO: 153 - protein, 11 aa, Synthetic construct [sequence omitted]

SEQ ID NO: 154 - protein, 13 aa, Synthetic construct
  REGION 1..13: note = 1-45 Light Chain CDR3
CQSFDRSLDGYAF

SEQ ID NO: 155 - protein, 13 aa, Synthetic construct
  REGION 1..13: note = 1-47 Light Chain CDR3
CHSYDRSLGGYVF

SEQ ID NO: 156 - protein, 10 aa, Synthetic construct [sequence omitted]

SEQ ID NO: 157 - protein, 12 aa, Synthetic construct
  REGION 1..12: note = 2-1 Light Chain CDR3
CHSYDRTLREVF

SEQ ID NO: 158 - protein, 13 aa, Synthetic construct
  REGION 1..13: note = 1-2 Heavy Chain CDR3
ARAAYDSLTYFEF

SEQ ID NO: 159 - protein, 21 aa, Synthetic construct
  REGION 1..21: note = hMPV F (A1); hMPV F (B1)
EVTAIKNALKKTNEAVSTLGN

SEQ ID NO: 160 - protein, 21 aa, Synthetic construct
  REGION 1..21: note = hMPV F (A2)
EVTAIKNALKTTNEAVSTLGN

SEQ ID NO: 161 - protein, 21 aa, Synthetic construct
  REGION 1..21: note = hMPV F (B2)
EVNAIKGALKTTNEAVSTLGN

SEQ ID NO: 162 - protein, 21 aa, Synthetic construct
  REGION 1..21: note = RSV F (A2)
EVNKIKSALLSTNKAVVSLSN

SEQ ID NO: 163 - protein, 21 aa, Synthetic construct
  REGION 1..21: note = RSV F (B)
EVNKIKNALQLTNKAVVSLSN

SEQ ID NO: 164 - protein, 21 aa, Synthetic construct
  REGION 1..21: note = hMPV F (A1);hMPV F (A2); hMPV F (B1)
GVRVLATAVRELKDFVSKNLT

SEQ ID NO: 165 - protein, 21 aa, Synthetic construct
  REGION 1..21: note = hMPV F (B2)
GVRVLATAVRELKEFVSKNLT

SEQ ID NO: 166 - protein, 21 aa, Synthetic construct
  REGION 1..21: note = RSV F (A2)
GVSVLTSKVLDLKNYIDKQLL

SEQ ID NO: 167 - protein, 21 aa, Synthetic construct
  REGION 1..21: note = RSV F (B)
GVSVLTSRVLDLKNYINNQLL

SEQ ID NO: 168 - protein, 21 aa, Synthetic construct
  REGION 1..21: note = hMPV F (A1); hMPV F (A2); hMPV F (B1)
ELARAVSNMPTSAGQIKLMLE

SEQ ID NO: 169 - protein, 21 aa, Synthetic construct
  REGION 1..21: note = hMPV F (B2)
ELARAVSYMPTSAGQIKLMLE

SEQ ID NO: 170 - protein, 21 aa, Synthetic construct
  REGION 1..21: note = RSV F (A2)
ELLSLINDMPITNDQKKLMSN

SEQ ID NO: 171 - protein, 21 aa, Synthetic construct
  REGION 1..21: note = RSV F (B)
ELLSLINDMPITNDQKKLMSS

(19) United States
(12) Patent Application Publication
(10) Pub. No.: US 2025/0296987 A1
(43) Pub. Date: Sep. 25, 2025

(54) RSV/HMPV CROSS-REACTIVE ANTIBODIES

(71) Applicant: VANDERBILT UNIVERSITY, Nashville, TN (US)

(72) Inventors: Alexandra Abu-Shmais, Nashville, TN (US); Ivelin Georgiev, Nashville, TN (US); Parastoo Amlashi, Nashville, TN (US)

(21) Appl. No.: 19/085,788

(22) Filed: Mar. 20, 2025

Related U.S. Application Data
(60) Provisional application No. 63/567,554, filed on Mar. 20, 2024.

(51) Int. Cl. C07K 16/10; A61K 39/00

(52) U.S. Cl. CPC C07K 16/1027; A61K 2039/505; C07K 2317/24; C07K 2317/33

(57) ABSTRACT

The present disclosure relates to cross-reactive RSV/hMPV antibodies, RSV/hMPV antibody compositions, and methods of use thereof.

### CROSS-REFERENCE TO RELATED APPLICATION

This U.S. utility application claims priority to, and the benefit of, U.S. Provisional Patent Application No. 63/567,554, filed Mar. 20, 2024, which is incorporated by reference herein in its entirety.

### STATEMENT REGARDING FEDERALLY SPONSORED RESEARCH

This invention was made with Government Support under Grant No. AI175245 awarded by the National Institutes of Health. The Government has certain rights in the invention.

### REFERENCE TO SEQUENCE LISTING

The sequence listing submitted on Mar. 20, 2025, as an .XML file entitled “10644-188US1-ST26” created on Mar. 13, 2025, and having a file size of 221,759 bytes is hereby incorporated by reference pursuant to 37 C.F.R. § 1.52(e)(5).

### FIELD

The present disclosure relates to cross-reactive RSV/hMPV antibodies, RSV/hMPV antibody compositions, and methods of use thereof.

### BACKGROUND

Human respiratory syncytial virus (RSV) and human metapneumovirus (hMPV) are worldwide, endemic respiratory pathogens of the Pneumoviridae family. Representing non-segmented negative-strand RNA viruses, RSV and hMPV induce severe and lethal bronchiolitis and pneumonia among particularly susceptible populations, most notably infantile, geriatric, and immunocompromised, with RSV being a leading cause of lower respiratory tract infection-associated hospitalization and mortality in children under 5 years of age. A turbulent history of disease enhancement following RSV vaccination has only recently been met with clinical success in the advancement of effective prophylactic strategies leveraging structure-based vaccine design and neutralizing antibodies with extended half-lives. Currently, there are no approved therapeutic or prophylactic options against hMPV infection. The antibodies, compositions, and methods disclosed herein address the need for improved RSV and hMPV antibodies.

The compounds, compositions, and methods disclosed herein address these and other needs.

### SUMMARY

The present disclosure provides a recombinant antibody or a composition thereof, wherein the recombinant antibody is reactive to respiratory syncytial virus (RSV) and/or human metapneumovirus (hMPV). The present disclosure also provides methods using a recombinant antibody or a composition thereof, wherein the recombinant antibody is reactive to RSV and/or hMPV.

In some aspects, disclosed herein is a recombinant antibody comprising a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, and SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, and SEQ ID NO: 136.

In some embodiments, the heavy chain comprises SEQ ID NO: 11 and the light chain comprises SEQ ID NO: 12. In some embodiments, the heavy chain comprises SEQ ID NO: 13 and the light chain comprises SEQ ID NO: 14. In some embodiments, the heavy chain comprises SEQ ID NO: 15 and the light chain comprises SEQ ID NO: 16. In some embodiments, the heavy chain comprises SEQ ID NO: 17 and the light chain comprises SEQ ID NO: 18. In some embodiments, the heavy chain comprises SEQ ID NO: 19 and the light chain comprises SEQ ID NO: 20. In some embodiments, the heavy chain comprises SEQ ID NO: 71 and the light chain comprises SEQ ID NO: 72. In some embodiments, the heavy chain comprises SEQ ID NO: 87 and the light chain comprises SEQ ID NO: 88. In some embodiments, the heavy chain comprises SEQ ID NO: 103 and the light chain comprises SEQ ID NO: 104. In some embodiments, the heavy chain comprises SEQ ID NO: 119 and the light chain comprises SEQ ID NO: 120. In some embodiments, the heavy chain comprises SEQ ID NO: 135 and the light chain comprises SEQ ID NO: 136.

In some embodiments, the heavy chain of any preceding aspect comprises a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143; a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, or SEQ ID NO: 158.

In some embodiments, the light chain of any preceding aspect comprises a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146; a CDR2 comprising amino acid sequences DNT, LDR, RAS, GNN, SEQ ID NO: 83, SEQ ID NO: 99, SEQ ID NO: 115, or SEQ ID NO: 131; and a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, or SEQ ID NO: 157.

In some embodiments, the recombinant antibody of any preceding aspect further comprises a heavy chain CDR1, CDR2, and CDR3 and a light chain CDR1, CDR2, and CDR3 selected from the group consisting of SEQ ID NO: 45, SEQ ID NO: 54, SEQ ID NO: 59, SEQ ID NO: 46, SEQ ID NO: 60, and amino acid sequence DNT; SEQ ID NO: 47, SEQ ID NO: 55, SEQ ID NO: 61, SEQ ID NO: 48, SEQ ID NO: 62, and amino acid sequence LDR; SEQ ID NO: 49, SEQ ID NO: 56, SEQ ID NO: 63, SEQ ID NO: 50, SEQ ID NO: 64, and amino acid sequence RAS; SEQ ID NO: 51, SEQ ID NO: 57, SEQ ID NO: 65, SEQ ID NO: 52, SEQ ID NO: 66, and amino acid sequence GNN; SEQ ID NO: 53, SEQ ID NO: 58, SEQ ID NO: 67, SEQ ID NO: 52, SEQ ID NO: 68, and amino acid sequence GNN; SEQ ID NO: 79, SEQ ID NO: 80, SEQ ID NO: 81, SEQ ID NO: 82, SEQ ID NO: 83, and SEQ ID NO: 84; SEQ ID NO: 95, SEQ ID NO: 96, SEQ ID NO: 97, SEQ ID NO: 98, SEQ ID NO: 99, and SEQ ID NO: 100; SEQ ID NO: 111, SEQ ID NO: 112, SEQ ID NO: 113, SEQ ID NO: 114, SEQ ID NO: 115, and SEQ ID NO: 116; SEQ ID NO: 127, SEQ ID NO: 128, SEQ ID NO: 129, SEQ ID NO: 130, SEQ ID NO: 131, and SEQ ID NO: 132; and SEQ ID NO: 143, SEQ ID NO: 144, SEQ ID NO: 145, SEQ ID NO: 146, SEQ ID NO: 147, and SEQ ID NO: 115.

In some embodiments, the recombinant antibody comprises SEQ ID NO: 45, SEQ ID NO: 54, SEQ ID NO: 59, SEQ ID NO: 46, SEQ ID NO: 60, and amino acid sequence DNT.

In some embodiments, the recombinant antibody comprises SEQ ID NO: 47, SEQ ID NO: 55, SEQ ID NO: 61, SEQ ID NO: 48, SEQ ID NO: 62, and amino acid sequence LDR.

In some embodiments, the recombinant antibody comprises SEQ ID NO: 49, SEQ ID NO: 56, SEQ ID NO: 63, SEQ ID NO: 50, SEQ ID NO: 64, and amino acid sequence RAS.

In some embodiments, the recombinant antibody comprises SEQ ID NO: 51, SEQ ID NO: 57, SEQ ID NO: 65, SEQ ID NO: 52, SEQ ID NO: 66, and amino acid sequence GNN.

In some embodiments, the recombinant antibody comprises SEQ ID NO: 53, SEQ ID NO: 58, SEQ ID NO: 67, SEQ ID NO: 52, SEQ ID NO: 68, and amino acid sequence GNN.

In some embodiments, the recombinant antibody comprises SEQ ID NO: 79, SEQ ID NO: 80, SEQ ID NO: 81, SEQ ID NO: 82, SEQ ID NO: 83, and SEQ ID NO: 84.

In some embodiments, the recombinant antibody comprises SEQ ID NO: 95, SEQ ID NO: 96, SEQ ID NO: 97, SEQ ID NO: 98, SEQ ID NO: 99, and SEQ ID NO: 100.

In some embodiments, the recombinant antibody comprises SEQ ID NO: 111, SEQ ID NO: 112, SEQ ID NO: 113, SEQ ID NO: 114, SEQ ID NO: 115, and SEQ ID NO: 116.

In some embodiments, the recombinant antibody comprises SEQ ID NO: 127, SEQ ID NO: 128, SEQ ID NO: 129, SEQ ID NO: 130, SEQ ID NO: 131, and SEQ ID NO: 132.

In some embodiments, the recombinant antibody comprises SEQ ID NO: 143, SEQ ID NO: 144, SEQ ID NO: 145, SEQ ID NO: 146, SEQ ID NO: 147, and SEQ ID NO: 115.

In some embodiments, the recombinant antibody of any preceding aspect further comprises a heavy chain CDR selected from SEQ ID NO: 148, SEQ ID NO: 149, SEQ ID NO: 150, SEQ ID NO: 151, SEQ ID NO: 152, or SEQ ID NO: 158.

In some embodiments, the recombinant antibody of any preceding aspect further comprises a light chain CDR selected from SEQ ID NO: 153, SEQ ID NO: 154, SEQ ID NO: 155, SEQ ID NO: 156, or SEQ ID NO: 157.

In some embodiments, the recombinant antibody comprises an antigen-binding site to human respiratory syncytial virus (RSV). In some embodiments, the recombinant antibody comprises an antigen-binding site to human metapneumovirus (hMPV). In some embodiments, the recombinant antibody comprises an antigen-binding site to RSV and hMPV.

In some aspects, disclosed herein is a nucleic acid sequence encoding the recombinant antibody of any preceding aspect.

In some aspects, disclosed herein is an expression vector comprising the nucleic acid of any preceding aspect and/or an expression vector encoding the recombinant antibody of any preceding aspect.

In some aspects, disclosed herein is a cell comprising the nucleic acid of any preceding aspect, a cell expressing the expression vector of any preceding aspect, and/or a cell comprising the recombinant antibody of any preceding aspect.

In some aspects, disclosed herein is a method of treating a respiratory infection in a subject in need thereof, the method comprising administering to the subject a recombinant antibody composition, wherein the recombinant antibody composition comprises a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, or SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, or SEQ ID NO: 136, wherein the heavy chain comprises a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143; a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, or SEQ ID NO: 158; and wherein the light chain comprises a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146; a CDR2 comprising amino acid sequences DNT, LDR, RAS, GNN, SEQ ID NO: 83, SEQ ID NO: 99, SEQ ID NO: 115, or SEQ ID NO: 131; and a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, or SEQ ID NO: 157.

### BRIEF DESCRIPTION OF FIGURES

The accompanying figures, which are incorporated in and constitute a part of this specification, illustrate several aspects described below.

FIGS. 1A and 1B show the identification and characterization of RSV/hMPV cross-reactive antibodies. FIG. 1A shows the LIBRA-seq predicted RSV and hMPV specific B cells. Each dot indicates an individual B cell. Max RSV A/RSV B LIBRA-seq score on the x-axis, max hMPV A/hMPV B LIBRA-seq score on the y-axis. Open data points (∘) were selected for further characterization. FIG. 1B shows the ELISA binding of recombinantly produced antibodies against RSV and hMPV prefusion F trimer, calculated as absorbance at 450 nm. Experiments were performed in technical and biological duplicate.

FIGS. 2A, 2B, and 2C show the binding characteristics of RSV/hMPV cross-reactive mAbs. FIG. 2A shows the antibody-antibody competition binding to RSV and hMPV prefusion F trimer against control site specific antibodies. Percentage of binding of biotinylated antibody is shown as a heatmap from 0% (black) to 100% (white). Non-biotinylated competitor antibodies were coated first, and then biotinylated control mAbs were added to detect competition. Competition is calculated as the signal obtained for binding of the biotin-labelled reference antibody in the presence of the unlabeled antibody, expressed as a percentage of the binding of the reference antibody alone. FIG. 2B shows the epitope binning via BLI for binding of mAbs 20 and 5-1 to RSV and hMPV prefusion F trimer. Data indicates the percent binding of the second antibody in the presence of the first antibody, as compared to the second antibody alone. Percentage of binding is shown as a heatmap from 0% (black) to 100% (white). FIG. 2C shows the ELISA binding of germline reverted, recombinantly produced antibodies against RSV A and B and hMPV A and B prefusion F trimer, calculated as absorbance at 450 nm. ELISA area under the curve (AUC) shown as a heatmap from minimum (white) to maximum binding (purple).

FIGS. 3A and 3B show the neutralization potency of RSV/hMPV cross-reactive mAbs. FIG. 3A shows the antibody neutralization against RSV A2, RSV B1, hMPV A2, and hMPV B2 via PRNT. FIG. 3B shows the IC₅₀ values, expressed as a heatmap with strong neutralization (<0.1 μg/mL) shown in purple and weak/non neutralizing (>10 μg/mL) shown in light purple. Calculated by non-linear regression analysis by GraphPad Prism software. Neutralization assays were performed in technical triplicate; data are represented as mean±SD.

FIGS. 4A, 4B, 4C, 4D, and 4E show the 5-1 Fab binding to the prefusion hMPV F peptide at site II, V, and the glycan at Asn172. FIG. 4A shows the Front view and side view of the fit of hMPV F complex into a DeepEMhanced EM map at the contour level of 0.432. The global DeepEMhanced EM map was shown as a white transparent map with a single hMPV F protomer and Fab variable domain colored (hMPV F, blue; heavy chain variable domain, red; light chain variable domain, orange). FIG. 4B shows the overlay of the 5-1 epitope onto the defined antigenic sites of hMPV F revealing that 5-1 primarily interacts with residues in site II and V, with additional contacts within site Ø. FIG. 4C shows the atomic model of 5-1 and hMPV F interface with key residues highlighted as sticks. 5-1 and one hMPV F protomer are shown as cartoons. Oxygen atoms are colored red and Nitrogen atoms are colored blue. Partially modeled Asn-172 glycan is shown as deep color sticks. FIG. 4D shows the sequence conservation of the 5-1 epitope between hMPV F and RSV F with the epitope of 5-1 delineated in white. FIG. 4E shows the sequence alignment of the 5-1 epitope with four representative hMPV F sequences from A1, A2, B1, B2 subgroup and two representative RSV sequences from A2 and B subgroup. The conservation of each residue is described underneath and the 5-1 interacting residues are highlighted in red. The glycosylation site at Asn-172 is shown as a branch.

FIGS. 5A and 5B show the 5-1 Fab prophylaxis of 5-1 against RSV and hMPV challenge. Protective efficacy of 5-1 against A) RSV and B) hMPV replication in vivo. BALB/c mice were treated intraperitoneally with 10 mg/kg, 1 mg/kg, and 0.1 mg/kg of mAb 5-1 6 h prior to intranasal RSV and hMPV infection. Viral titers in the lung homogenates of BALB/c mice in each treatment group (n=5 mice per group, 5 females) were determined by plaque assay. n.s., not significant, Limit of detection (LOD) is indicated with a dashed line.

FIG. 6 shows the mAb binding to Hep2 cells. Images of representative mAbs staining of HEp-2 cells. Indirect immunofluorescence assay testing reactivity of RSV/hMPV mAbs in HEp-2 cells. Each mAb was tested at 1 and 10 μg/mL. Positivity scores were determined relative to positive (ANA+human serum) and negative (ANA−human serum) controls. DAPI staining (blue) was used to visualize nuclear DNA, goat anti-human Ig-FITC (green) staining notes Hep-2 cell reactivity. For all images, brightness was set to 150 and contrast was set to 100 using Photoshop.

FIGS. 7A and 7B show the 5-1 Fab binding to DS-Cav1. DS-Cav1 complexed with 5-1 at 10 μg/mL. Left at 30 nm, right at 50 nm.

FIG. 8 shows the hMPV and 5-1 Fab cryoEM dataset processing workflow. Representative micrographs, EM maps, computational programs and software from each step of the workflow are shown and labeled. The mask used for 3D classification is shown as a transparent purple surface.

FIGS. 9A, 9B, 9C, and 9D show the validation of the obtained hMPV F EM map. FIG. 9A shows the fitting of the DeepEMhanced EM map into the raw, unsharpened EM map. The raw, unsharpened EM map is shown as a transparent surface at the threshold of 0.0658. The DeepEMhanced EM map is shown as an opaque surface at the threshold of 0.431 with an individual hMPV F and Fab variable domain colored as indicated. FIG. 9B shows the surface of the raw unsharpened EM map was colored by local resolution at the threshold of 0.026. FIG. 9C shows the FSC curves and particle orientation distribution for the EM map from the final homogeneous refinement step. Top, FSC curves; Botton, particle orientation distribution. Horizon line in FSC curves corresponds to an FSC value of 0.143. FIG. 9D shows the binding interface between hMPV F-DsCavEs2-IPDS and 5-1 Fab. CryoEM map was shown as a transparent surface with the model fitted and colored.

FIG. 10 shows steric clashes between 5-1 and site-specific antibodies. 5-1 shows significant clashes with competing antibodies and little to no steric clashing with non-competing antibodies from FIG. 2. Selected antibodies are shown as transparent surface and 5-1 is shown as cartoon with the light and heavy chain colored as orange and red, respectively. 101 F and DS7 are modeled onto hMPV F trimers because of their close distance on native protomers.

FIGS. 11A, 11B, and 11C show the binding poses and epitope conservation of antibodies binding site V. FIG. 11A shows the modelling of site V antibodies with 5-1 Fab shows different binding poses on hMPV F. The quaternary antibody AM-14 was included for completeness. 5-1 is shown as opaque surface with the light and heavy chains colored as orange and red, respectively. Selected antibodies are modelled as transparent surface with the light and heavy chains colored as lavender and purple, respectively. FIG. 11B shows the antigenic footprints of 5-1 and site V antibodies target different epitopes inside site V and often bind residues beyond site V. FIG. 11C shows the comparison of epitopes based on sequence conservation reveals that sequence conservation did not solely determine the cross-neutralization properties of antibodies.

FIGS. 12A and 12B show the N-linked glycans and 5-1 Fab binding. FIG. 12A shows the front view (left) and top view (right) of the N-linked complex glycans on hMPV F trimers. Glycans shown as ticks. FIG. 12B shows the fit of the 5-1 Fab onto the modeled hMPV F trimers shows the light chain of 5-1 inserts into the cleft between Asn57-glycan and Asn172-glycan without clashes with Asn172-Glycan.

FIGS. 13A, 13B, and 13C shows the identification of cross-reactive antibodies from healthy donors using LIBRA-seq. FIG. 13A shows the LIBRA-seq predicted RSV and hMPV specific B cells. Each point indicates individual B cells. Max RSV prefusion and RSV postfusion LIBRA-seq score on the Y axis. Triangles were selected for further characterization. FIG. 13B shows the ELISA binding of recombinantly produced antibodies against RSV A prefusion F trimer and hMPV B prefusion F trimer, calculated as absorbance at 450 nm. ELISA area under the curve (AUC) is shown as a heatmap from minimum (white) to maximum (purple) binding. FIG. 13C shows the ELISA binding of cross-reactive antibodies against D280N point mutation hMPV variants, calculated as absorbance at 450 nm with the accompanied AUC shown as a heatmap from minimum (white) to maximum (purple) binding.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100%, so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of a viral infection), during early onset (e.g., upon initial signs and symptoms of the viral infection), or after an established development of the viral infection.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcϵRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

As used herein, a “recombinant antibody” refers to an antibody produced through genetic manipulation or engineering techniques, rather than by traditional or natural immunization methods. Said genetic manipulation includes but is not limited to isolation of antibody genes, cloning of antibody genes inside an expression vector, expression of antibody genes inside a host organism, such as for example bacterial cells, yeast cells, and/or mammalian cells, and purification of the recombinant antibodies from the host organisms. It should be understood that following purification of a recombinant antibody, additional modifications can be incorporated into said recombinant antibody to enhance its properties.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

A “vaccine” refers to a biological preparation that provides active acquired immunity to a particular infectious diseases caused by a virus, or any other microorganisms. Vaccines typically comprise an agent or several agents, also referred to as antigens, resembling the disease-causing microorganism and is often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage. In some embodiments, a therapeutic composition comprises a vaccine and/or other agents (including, but not limited to adjuvants, preservatives, stabilizers, salts, additives, and/or combinations thereof).

An “epitope” or “antigenic determinant” refer to the part of an antigen, a molecular structure, or foreign particulate that can bind to a specific antibody or T-cell receptor. The presence of antigens or epitopes of antigens within a host can illicit an immune response.

An “antigen” refers to a molecule, moiety, foreign particulate matter, or an allergen that can bind to a specific antibody or T cell receptor. The presence of antigens within a host can illicit an immune response against said molecule, moiety, foreign particulate matter, or allergen.

A “paratope” refers to the portion of an antibody which recognizes and binds to an antigen. In general, the paratope is a small region at the tip of the antibody's antigen-binding fragment and contains parts of the antibody's heavy and light chains. Each paratope comprises six complementarity determining regions (CDRs)—three from each of the light and heavy chains. It should be understood that paratope and antigen-binding site can be used interchangeably.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

A “vector” or an “expression vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In other embodiments, the vector is a lipid nanoparticle. Lipid nanoparticles can be used to deliver mRNA to a host cell for expression of the mRNA in the host cell.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

### Antibodies and Antibody Compositions

The respiratory syncytial virus (RSV) and human metapneumovirus (hMPV) are major effectors in causing upper and lower respiratory disease leading to high hospitalization rates in infants, significant morbidity in children and adults, and excess mortality in elderly. RSV and/or hMPV vaccine development to prevent infections of these virus have been the focus of many research efforts, however, there remains several limitations and challenges to developing said vaccines. Current challenges to RSV vaccine development include but are not limited to early age of RSV infection, capacity of RSV to evade innate immunity, failure of RSV-induced adaptive immunity to prevent re-infection, history of RSV vaccine-enhanced disease, and lack of an animal model fully permissive to human RSV infection. Regarding hMPV vaccine, there are currently no such vaccines available to the public. The present disclosure provides an antibody composition to target, prevent, and/or treat RSV and/or hMPV infections.

The present disclosure provides a recombinant antibody or a composition thereof, wherein the recombinant antibody is reactive to respiratory syncytial virus (RSV) and/or human metapneumovirus (hMPV). The present disclosure also provides a recombinant antibody or a composition thereof, comprising a cross-reactive antibody that binds and/or reacts to RSV and hMPV. As used herein, a “cross-reactive antibody” refers to an antibody that is developed against at least one antigen but recognizes and binds to at least two antigens that have similar structural regions, such that the at least two antigen comprise identical or very similar epitopes. The benefits of a cross-reactive antibody, such as a cross-reactive RSV/hMPV antibody, include, but are not limited to broader immunity against pathogens or related pathogens. In some embodiments, the recombinant antibody of any aspect disclosed herein is a cross-reactive antibody.

In some aspects, disclosed herein is a recombinant antibody comprising a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, and SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, and SEQ ID NO: 136; or a sequence at least 60% identical (for example, at least 60% identical, at least 70% identical, at least 80% identical, at least 90% identical, or at least 95%) thereof.

In some embodiments, the heavy chain comprises SEQ ID NO: 11 and the light chain comprises SEQ ID NO: 12. In some embodiments, the heavy chain comprises SEQ ID NO: 13 and the light chain comprises SEQ ID NO: 14. In some embodiments, the heavy chain comprises SEQ ID NO: 15 and the light chain comprises SEQ ID NO: 16. In some embodiments, the heavy chain comprises SEQ ID NO: 17 and the light chain comprises SEQ ID NO: 18. In some embodiments, the heavy chain comprises SEQ ID NO: 19 and the light chain comprises SEQ ID NO: 20. In some embodiments, the heavy chain comprises SEQ ID NO: 71 and the light chain comprises SEQ ID NO: 72. In some embodiments, the heavy chain comprises SEQ ID NO: 87 and the light chain comprises SEQ ID NO: 88. In some embodiments, the heavy chain comprises SEQ ID NO: 103 and the light chain comprises SEQ ID NO: 104. In some embodiments, the heavy chain comprises SEQ ID NO: 119 and the light chain comprises SEQ ID NO: 120. In some embodiments, the heavy chain comprises SEQ ID NO: 135 and the light chain comprises SEQ ID NO: 136.

In some embodiments, the heavy chain of any preceding aspect comprises a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143; a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, SEQ ID NO: 158, or a fragment thereof; or a sequence at least 60% identical (for example, at least 60% identical, at least 70% identical, at least 80% identical, at least 90% identical, or at least 95%) thereof.

In some embodiments, the light chain of any preceding aspect comprises a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146; a CDR2 comprising amino acid sequences DNT, LDR, RAS, GNN, SEQ ID NO: 83, SEQ ID NO: 99, SEQ ID NO: 115, or SEQ ID NO: 131; and a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, SEQ ID NO: 157, or a fragment thereof; or a sequence at least 60% identical (for example, at least 60% identical, at least 70% identical, at least 80% identical, at least 90% identical, or at least 95%) thereof.

In some embodiments, the recombinant antibody of any preceding aspect further comprises a heavy chain CDR1, CDR2, and CDR3 and a light chain CDR1, CDR2, and CDR3 selected from the group consisting of SEQ ID NO: 45, SEQ ID NO: 54, SEQ ID NO: 59, SEQ ID NO: 46, SEQ ID NO: 60, and amino acid sequence DNT; SEQ ID NO: 47, SEQ ID NO: 55, SEQ ID NO: 61, SEQ ID NO: 48, SEQ ID NO: 62, and amino acid sequence LDR; SEQ ID NO: 49, SEQ ID NO: 56, SEQ ID NO: 63, SEQ ID NO: 50, SEQ ID NO: 64, and amino acid sequence RAS; SEQ ID NO: 51, SEQ ID NO: 57, SEQ ID NO: 65, SEQ ID NO: 52, SEQ ID NO: 66, and amino acid sequence GNN; SEQ ID NO: 53, SEQ ID NO: 58, SEQ ID NO: 67, SEQ ID NO: 52, SEQ ID NO: 68, and amino acid sequence GNN; SEQ ID NO: 79, SEQ ID NO: 80, SEQ ID NO: 81, SEQ ID NO: 82, SEQ ID NO: 83, and SEQ ID NO: 84; SEQ ID NO: 95, SEQ ID NO: 96, SEQ ID NO: 97, SEQ ID NO: 98, SEQ ID NO: 99, and SEQ ID NO: 100; SEQ ID NO: 111, SEQ ID NO: 112, SEQ ID NO: 113, SEQ ID NO: 114, SEQ ID NO: 115, and SEQ ID NO: 116; SEQ ID NO: 127, SEQ ID NO: 128, SEQ ID NO: 129, SEQ ID NO: 130, SEQ ID NO: 131, and SEQ ID NO: 132; and SEQ ID NO: 143, SEQ ID NO: 144, SEQ ID NO: 145, SEQ ID NO: 146, SEQ ID NO: 147, and SEQ ID NO: 115; or a sequence at least 60% identical (for example, at least 60% identical, at least 70% identical, at least 80% identical, at least 90% identical, or at least 95%) thereof.

In some embodiments, the recombinant antibody comprises SEQ ID NO: 45, SEQ ID NO: 54, SEQ ID NO: 59, SEQ ID NO: 46, SEQ ID NO: 60, and amino acid sequence DNT; or a sequence at least 60% identical (for example, at least 60% identical, at least 70% identical, at least 80% identical, at least 90% identical, or at least 95%) thereof.

In some embodiments, the recombinant antibody comprises SEQ ID NO: 47, SEQ ID NO: 55, SEQ ID NO: 61, SEQ ID NO: 48, SEQ ID NO: 62, and amino acid sequence LDR; or a sequence at least 60% identical (for example, at least 60% identical, at least 70% identical, at least 80% identical, at least 90% identical, or at least 95%) thereof.

In some embodiments, the recombinant antibody comprises SEQ ID NO: 49, SEQ ID NO: 56, SEQ ID NO: 63, SEQ ID NO: 50, SEQ ID NO: 64, and amino acid sequence RAS; or a sequence at least 60% identical (for example, at least 60% identical, at least 70% identical, at least 80% identical, at least 90% identical, or at least 95%) thereof.

In some embodiments, the recombinant antibody comprises SEQ ID NO: 51, SEQ ID NO: 57, SEQ ID NO: 65, SEQ ID NO: 52, SEQ ID NO: 66, and amino acid sequence GNN; or a sequence at least 60% identical (for example, at least 60% identical, at least 70% identical, at least 80% identical, at least 90% identical, or at least 95%) thereof.

In some embodiments, the recombinant antibody comprises SEQ ID NO: 53, SEQ ID NO: 58, SEQ ID NO: 67, SEQ ID NO: 52, SEQ ID NO: 68, and amino acid sequence GNN; or a sequence at least 60% identical (for example, at least 60% identical, at least 70% identical, at least 80% identical, at least 90% identical, or at least 95%) thereof.

In some embodiments, the recombinant antibody comprises SEQ ID NO: 79, SEQ ID NO: 80, SEQ ID NO: 81, SEQ ID NO: 82, SEQ ID NO: 83, and SEQ ID NO: 84; or a sequence at least 60% identical (for example, at least 60% identical, at least 70% identical, at least 80% identical, at least 90% identical, or at least 95%) thereof.

In some embodiments, the recombinant antibody comprises SEQ ID NO: 95, SEQ ID NO: 96, SEQ ID NO: 97, SEQ ID NO: 98, SEQ ID NO: 99, and SEQ ID NO: 100; or a sequence at least 60% identical (for example, at least 60% identical, at least 70% identical, at least 80% identical, at least 90% identical, or at least 95%) thereof.

In some embodiments, the recombinant antibody comprises SEQ ID NO: 111, SEQ ID NO: 112, SEQ ID NO: 113, SEQ ID NO: 114, SEQ ID NO: 115, and SEQ ID NO: 116; or a sequence at least 60% identical (for example, at least 60% identical, at least 70% identical, at least 80% identical, at least 90% identical, or at least 95%) thereof.

In some embodiments, the recombinant antibody comprises SEQ ID NO: 127, SEQ ID NO: 128, SEQ ID NO: 129, SEQ ID NO: 130, SEQ ID NO: 131, and SEQ ID NO: 132; or a sequence at least 60% identical (for example, at least 60% identical, at least 70% identical, at least 80% identical, at least 90% identical, or at least 95%) thereof.

In some embodiments, the recombinant antibody comprises SEQ ID NO: 143, SEQ ID NO: 144, SEQ ID NO: 145, SEQ ID NO: 146, SEQ ID NO: 147, and SEQ ID NO: 115; or a sequence at least 60% identical (for example, at least 60% identical, at least 70% identical, at least 80% identical, at least 90% identical, or at least 95%) thereof.

In some embodiments, the recombinant antibody of any preceding aspect further comprises a heavy chain CDR selected from SEQ ID NO: 148, SEQ ID NO: 149, SEQ ID NO: 150, SEQ ID NO: 151, SEQ ID NO: 152, or SEQ ID NO: 158; or a sequence at least 60% identical (for example, at least 60% identical, at least 70% identical, at least 80% identical, at least 90% identical, or at least 95%) thereof.

In some embodiments, the recombinant antibody of any preceding aspect further comprises a light chain CDR selected from SEQ ID NO: 153, SEQ ID NO: 154, SEQ ID NO: 155, SEQ ID NO: 156, or SEQ ID NO: 157; or a sequence at least 60% identical (for example, at least 60% identical, at least 70% identical, at least 80% identical, at least 90% identical, or at least 95%) thereof.

In some embodiments, the recombinant antibody further comprises any combination of heavy chain CDRs of any preceding aspect. In some embodiments, the recombinant antibody further comprises any combination of light chain CDRs of any preceding aspect.

In some embodiments, the recombinant antibody comprises an antigen-binding site to human respiratory syncytial virus (RSV). In some embodiments, the recombinant antibody comprises an antigen-binding site to human metapneumovirus (hMPV). In some embodiments, the recombinant antibody comprises an antigen-binding site to RSV and hMPV.

LIBRA-seq (Linking B Cell Receptor to Antigen specificity through sequencing) is developed to simultaneously recover both antigen specificity and paired heavy and light chain BCR sequence. LIBRA-seq is a next-generation sequencing-based readout for BCR-antigen binding interactions that utilizes oligonucleotides (oligos) conjugated to recombinant antigens. Antigen barcodes are recovered during paired-chain BCR sequencing experiments and bioinformatically mapped to single cells. The LIBRA-seq method was applied to PBMC samples from two HIV-infected subjects, and from these, HIV- and influenza-specific antibodies were successfully identified, including both known and novel broadly neutralizing antibody (bNAb) lineages. LIBRA-seq is high-throughput, scalable, and applicable to many targets. This single, integrated assay enables the mapping of monoclonal antibody sequences to panels of diverse antigens theoretically unlimited in number and facilitates the rapid identification of cross-reactive antibodies that serves as therapeutics or vaccine templates.

Following a LIBRA-seq experiment, there are 2 resulting pairs of FASTQ files: (1) B cell receptor libraries (containing heavy and light chain contigs), and (2) antigen barcode libraries (containing antigen-identifying DNA barcode sequences from the antigen screening library). In some embodiments, it should be understood that the methods described herein are for uniting the information from these two sequencing libraries. Accordingly, in some embodiments, the above noted step of removing a sequence lacking the cell barcode, the UMI, or the antigen barcode is for removing a sequence from the antigen barcode library lacking the cell barcode, the UMI, or the antigen barcode. The processing serves two purposes: (1) quality control and annotation of sequenced reads, and (2) identification of binding signal from the annotated sequenced reads. Before the following steps are carried out, the BCR libraries are processed in order to determine the list of cell barcodes that have a VDJ sequence.

The present disclosure incorporates by reference Georgiev et al (PCT/US20/49330) for its teaching of using LIBRA-seq to identify antigen binding specificity of antibodies. The present disclosure also incorporates by reference Georgiev et al (63/588,443) for its teaching of using LIBRA-seq systems and methods for simultaneous detection of antigens and ligands.

In some aspects, disclosed herein is a nucleic acid sequence encoding the recombinant antibody of any preceding aspect.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

In some aspects, disclosed herein is an expression vector comprising the nucleic acid of any preceding aspect and/or an expression vector encoding the recombinant antibody of any preceding aspect.

In some embodiments, the expression vector includes, but is not limited to a viral vector (such as for example a retroviral vector), an adenoviral vector, an adeno-associated viral vector, a large payload vector, a nanoparticle, or an extracellular vesicle.

### Retroviral Vectors

A retrovirus is an animal virus belonging to the virus family of Retroviridae, including any types, subfamilies, genus, or tropisms. Retroviral vectors, in general, are described by Verma, I.M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson., Curr Opin Mol Ther 5:633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

In some aspects, disclosed herein is a cell comprising the nucleic acid of any preceding aspect, a cell expressing the expression vector of any preceding aspect, and/or a cell comprising the recombinant antibody of any preceding aspect. In some embodiments, the cell is a prokaryotic cell, including but not limited to a bacterial cell such, as for example Escherichia coli (E. coli) bacterium. In some embodiments, the cell is a eukaryotic cell.

### Methods of Using an Antibody or a Composition Thereof

The present disclosure provides methods using a recombinant antibody or a composition thereof, wherein the antibody is reactive to RSV and/or hMPV.

In some aspects, disclosed herein is a method of treating a respiratory infection in a subject in need thereof, the method comprising administering to the subject a recombinant antibody composition comprising a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, and SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, and SEQ ID NO: 136; or a sequence at least 60% identical (for example, at least 60% identical, at least 70% identical, at least 80% identical, at least 90% identical, or at least 95%) thereof.

In some aspects, disclosed herein is a method of treating a respiratory infection in a subject in need thereof, the method comprising administering to the subject a recombinant antibody composition, wherein the recombinant antibody composition comprises a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, or SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, or SEQ ID NO: 136, wherein the heavy chain comprises a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143; a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, or SEQ ID NO: 158; and wherein the light chain comprises a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146; a CDR2 comprising amino acid sequences DNT, LDR, RAS, GNN, SEQ ID NO: 83, SEQ ID NO: 99, SEQ ID NO: 115, or SEQ ID NO: 131; and a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, or SEQ ID NO: 157; or a sequence at least 60% identical (for example, at least 60% identical, at least 70% identical, at least 80% identical, at least 90% identical, or at least 95%) thereof.

In some aspects, disclosed herein is a method of treating a respiratory infection in a subject in need thereof, the method comprising administering to the subject the recombinant antibody composition of any preceding aspect.

In some aspect, disclosed herein is a method of preventing a respiratory infection in a subject in need thereof, the method comprising administering to the subject the recombinant antibody composition of any preceding aspect.

In some embodiments, the antibody composition comprises the heavy chain CDR of any preceding aspect. In some embodiments, the recombinant antibody composition comprises the light chain CDR of any preceding aspect.

In some embodiments, the respiratory infection is caused by an RSV. In some embodiments, the respiratory infection is caused by a hMPV. In some embodiments, the respiratory infection is caused by an RSV and a hMPV.

The recombinant antibody composition may be administered in such amounts, time, and route deemed necessary in order to achieve the desired result. The exact amount of the recombinant antibody composition will vary from subject to subject, depending on the species, age, and general condition of the subject, the severity of the RSV and/or hMPV, the particular recombinant antibody composition, its mode of administration, its mode of activity, and the like. The recombinant antibody composition is preferably formulated in dosage unit form for ease of administration and uniformity of dosage. It will be understood, however, that the total daily usage of the recombinant antibody composition will be decided by the attending physician within the scope of sound medical judgment. The specific therapeutically effective dose level for any particular subject will depend upon a variety of factors including the viral infection(s) being treated and the severity of the infection symptoms; the activity of the recombinant antibody composition employed; the specific recombinant antibody composition employed; the age, body weight, general health, sex and diet of the patient; the time of administration, route of administration, and rate of excretion of the specific recombinant antibody composition employed; the duration of the treatment; drugs used in combination or coincidental with the specific recombinant antibody composition employed; and like factors well known in the medical arts.

The recombinant antibody composition may be administered by any route. In some embodiments, the antibody composition is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the recombinant antibody composition (e.g., its stability in the environment of the subject's body), the condition of the subject (e.g., whether the subject is able to tolerate oral administration), etc.

The exact amount of recombinant antibody composition required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is a recombinant antibody composition of any preceding aspect and a pharmaceutically acceptable carrier selected from an excipient, a diluent, a salt, a buffer, a stabilizer, a lipid, an emulsion, a nanoparticle, and a cream. One or more active agents can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the subject is a mammal. In some embodiments, the subject is a human, such as for example a newborn, an infant, an adolescent, an elderly human, or an immunocompromised human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: A Potently Neutralizing and Protective Human Antibody Targeting Antigenic Site V on RSV and hMPV Fusion Glycoprotein

Human respiratory syncytial virus (RSV) and human metapneumovirus (hMPV) are frequent drivers of morbidity and mortality in susceptible populations, most often infantile, older adults, and immunocompromised. The primary target of neutralizing antibodies is the fusion (F) glycoprotein on the surface of the RSV and hMPV virion. As a result of the structural conservation between RSV and hMPV F, three antigenic regions are known to induce cross-neutralizing responses: sites III, IV, and V. Leveraging LIBRA-seq, we identify five RSV/hMPV cross-reactive human antibodies. One antibody, 5-1, potently neutralizes all tested viruses from the major subgroups of RSV and hMPV and provides protection against RSV and hMPV in a mouse challenge model. Structural analysis reveals that 5-1 utilizes an uncommon genetic signature to bind an epitope that spans sites Ø, II and V, defining a new mode of antibody cross-reactivity between RSV and hMPV F. These findings highlight the molecular and structural elements influencing RSV and hMPV cross-reactivity as well as the potential of antibody 5-1 for translational development.

The major target of neutralizing antibodies in human sera against RSV and hMPV infection is the fusion (F) glycoprotein on the surface of the virion. RSV/hMPV F is a trimeric type I transmembrane fusion protein responsible for mediating viral entry into host cells of the airway epithelium. Substantial conformational changes occur in F as it transitions from the metastable prefusion form to the stable postfusion form, and understanding of these structural rearrangements has enabled engineering of prefusion-stabilized F antigens. Stabilization of RSV and hMPV F in the prefusion state induces high neutralizing titers in experimentally inoculated animals and prefusion-stabilized RSV F serves as the backbone of the recently approved human RSV vaccines. Importantly, differential glycosylation patterns on the apex of RSV and hMPV F result in conformationally specific contributions towards the induction of neutralizing responses: RSV prefusion F epitopes are exceptionally immunogenic and invoke potently neutralizing antibodies, whereas pre- and post-fusion hMPV F stimulate comparable neutralizing responses. Antibody isolation and characterization efforts against RSV and hMPV have enabled extensive definition of the antigenic landscapes of RSV and hMPV F. The antigenic topology of RSV and hMPV F follows a synonymous nomenclature, with the major sites represented as site Ø through site V, as well as the more recently described site VI on RSV F. Antigenic sites Ø, V and VI are preserved exclusively on the prefusion conformations of the proteins, whereas sites I, II, III, and IV are exposed on the pre- and postfusion conformations.

Broadly reactive and neutralizing antibodies that recognize both RSV and hMPV have been described with varied breadth and potency of virus neutralization. Due to the structural conservation between RSV and hMPV F glycoproteins, three shared epitopes on F elicit cross-reactive antibody responses, despite low sequence identity (˜35%) 35: sites III, IV, and V. Site III is highly conserved between both viruses and a common target of cross-neutralizing antibodies encoded by IGHV3-11/IGHV3-21: IGLV1-40, a germline gene pairing reported to be enriched in infant and adult anti-RSV antibody repertoires recognizing site III. Low- and high-resolution structural analyses of site III and IV cross-reactive antibodies provide evidence that binding pose may influence cross-reactivity; however, the mode of antigenic recognition of a site V cross-neutralizing antibody remains unknown.

Leveraging LIBRA-seq (Linking B cell Receptor Sequence to Antigen Specificity by Sequencing), five RSV/hMPV cross-reactive antibodies were identified from human PBMC samples that showed high neutralization potencies against both RSV and hMPV that were comparable to virus-specific (RSV- or hMPV-only) antibodies in the literature, with one monoclonal antibody (mAb) 5-1 potently neutralizing the major subgroups of RSV and hMPV. We determined the epitope of 5-1 by single-particle cryo-EM using a prefusion-stabilized hMPV F with inter- and intra-protomer disulfide bonds and found that the binding site of 5-1 spans antigenic sites Ø, II and V on an individual protomer. Analysis of the interface identifies residues that are important for RSV and hMPV cross-neutralization. Finally, 5-1 showed robust protection in a mouse challenge model against both RSV and hMPV, therefore establishing this antibody as a prime candidate for further translational development.

### Results

### Isolation of RSV/hMPV Cross-Reactive Monoclonal Antibodies by LIBRA-Seq

To identify RSV/hMPV cross-reactive antibodies, previously reported LIBRA-seq datasets were searched that included prefusion-stabilized F glycoproteins from RSV A, RSV B, hMPV A, hMPV B, as well as control antigens. These B cells were bulk sorted from healthy donor PBMC samples, based on the expression of several markers: CD19⁺, IgG⁺, antigen⁺. After sequencing and computational filtering, a total of 27 B cells with positive signal (defined as a minimum LIBRA-seq score of 1) were isolated for at least one of the F glycoproteins belonging to both RSV and hMPV, while exhibiting low signal (defined as a LIBRA-seq score less than 1) for binding to control antigens.

### Epitope Mapping and In-Vitro Functional Properties

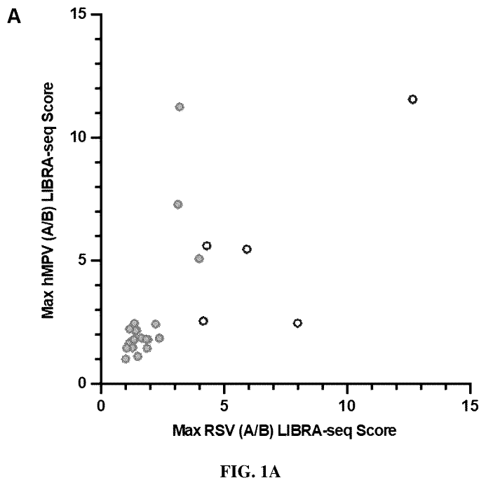

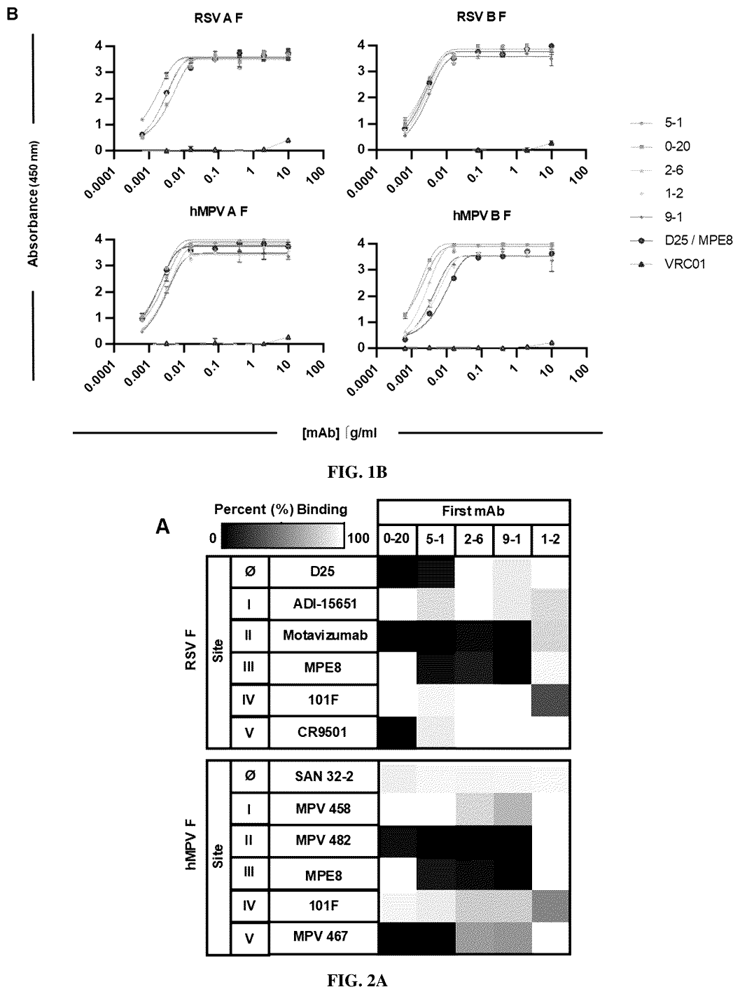

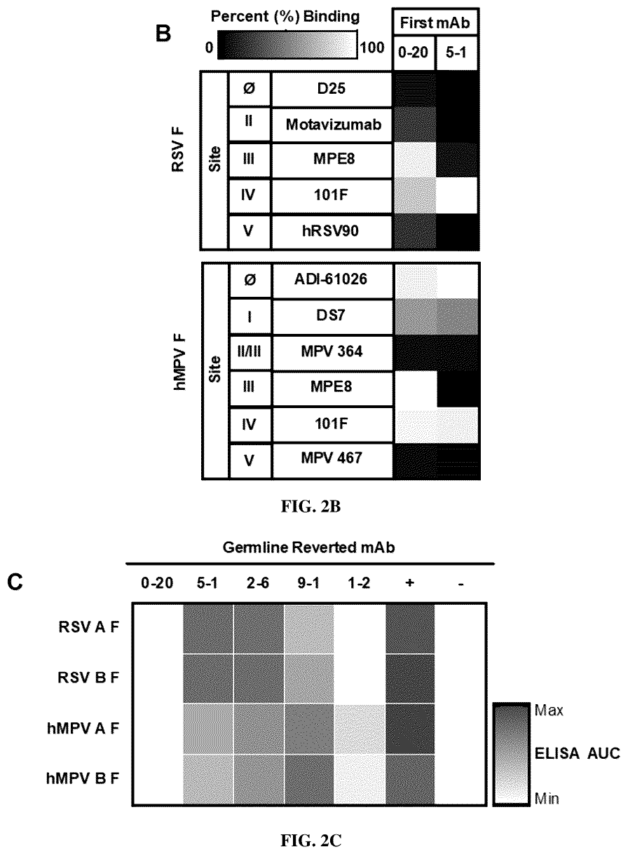

Five B cell receptor sequences from the analysis herein, corresponding to B cells with high LIBRA-seq scores of at least 1 or both RSV A/B and hMPV A/B, were produced recombinantly as IgG1 monoclonal antibodies (mAb) (FIG. 1A). Four of the five antibodies are encoded by gene segments belonging to the VH3 family, with two of the four using the archetypal IGHV3-11/3-21: IGLV1-40 of site III cross-reactive antibodies such as MPE8, 25P13, RSV199, and MxR. In contrast, mAb 5-1 leveraged a pairing not yet reported among RSV/hMPV cross-reactive B cells (Table 1). Predicted reactivity was confirmed via enzyme-linked immunosorbent assay (ELISA) (FIG. 1B). To investigate the antigenic binding sites of the cross-reactive mAbs, the antibodies were tested for competition ELISA binding against site-specific published antibodies with prefusion-stabilized RSV F and hMPV F protein antigens. Antibodies 2-6, 9-1, and 1-2 displayed consistent competition binding profiles on RSV and hMPV F proteins, mapping to sites III (2-6, 9-1) and IV (1-2). Intriguingly, mAb 5-1 strongly competed for binding to multiple sites on RSV prefusion F (sites Ø, II, III) and hMPV prefusion F (sites II, III, V). mAb 0-20 also strongly competed for binding to multiple sites on RSV prefusion F (sites Ø, II, V) and hMPV prefusion F (sites II and V) (FIG. 2A). Due to the unusual competition profiles of mAbs 5-1 and 0-20, epitope binning was conducted using competition biolayer interferometry (BLI). Individually, prefusion-stabilized RSV or hMPV F protein was loaded onto sensors before saturating with mAbs 5-1 or 0-20 followed by exposure to mAbs with known antigenic epitopes. Similar to their competition ELISA binding profile, mAb 5-1 competed with site Ø, II, III, and V mAbs on RSV, and II, III, and V on hMPV, while mAb 0-20 competed with site Ø, II, and V mAbs on RSV, and site II and V mAbs on hMPV (FIG. 2B).

To investigate whether cross-reactivity emerged as a result of somatic hypermutation, each candidate mAb was reverted to its germline sequence and tested binding to recombinant F antigens. While mAbs 9-1 and 2-6 both target site III, germline-reverted mAb 2-6 preferred binding to RSV F while germline-reverted mAb 9-1 preferred binding to hMPV F (FIG. 2C). Binding to both RSV and hMPV F was abrogated for the germline-reverted mAb 0-20, while mAb 5-1 and mAb 1-2 displayed preferential binding to RSV F and hMPV F, respectively (FIG. 2C).

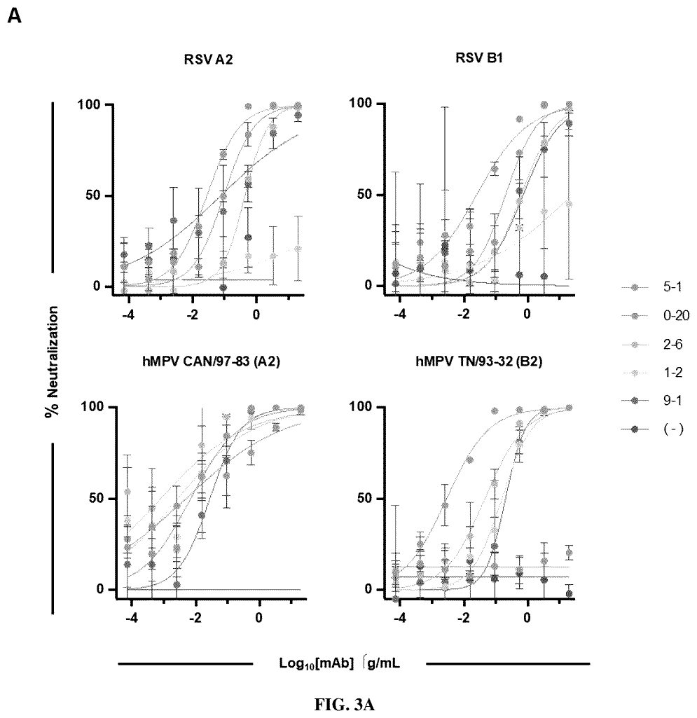

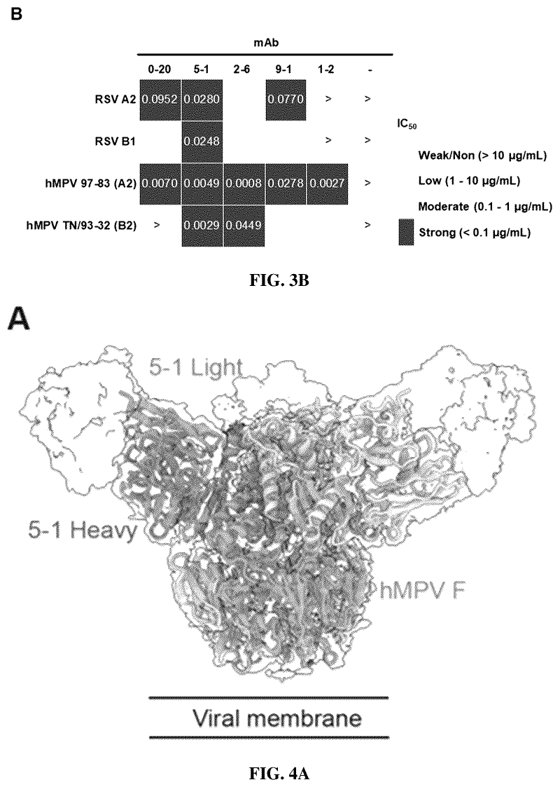

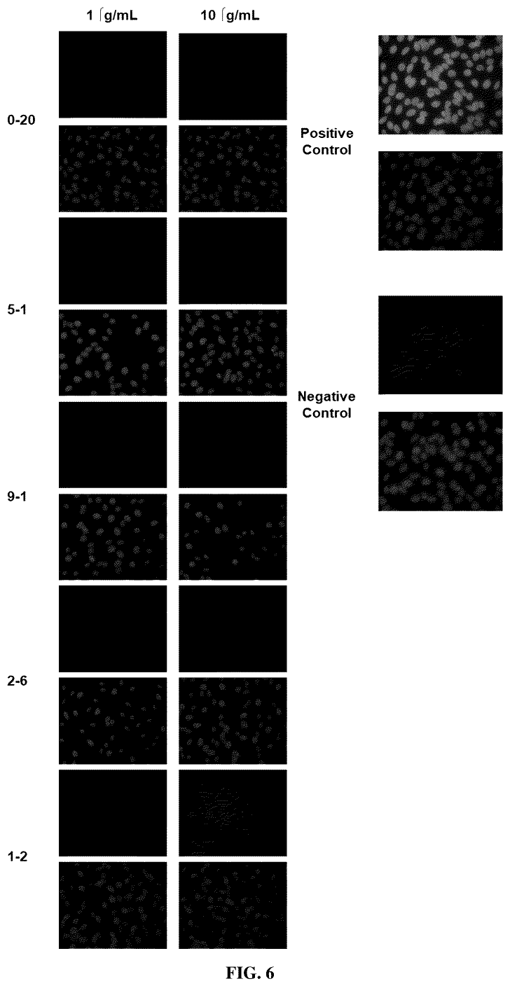

Antibody-virus neutralization potency was determined by plaque reduction neutralization test (PRNT) using live virus to inoculate cells. All candidate mAbs exhibited neutralization against at least one of the tested viruses representing the major antigenic groups of RSV and hMPV. Notably, while mAb 5-1 demonstrated higher neutralization potencies against hMPV compared to RSV viruses, this antibody exhibited strong neutralization against all viruses tested (IC₅₀ 0.0029-0.0280 μg/mL) (FIGS. 3A and 3B). To assess autoreactivity, binding to permeabilized HEp-2 cells was performed. At 1 μg/mL and 10 μg/mL, none of the antibodies displayed binding to HEp-2 cells (FIG. 6).

Structure of mAb 5-1 Complexed with hMPV F

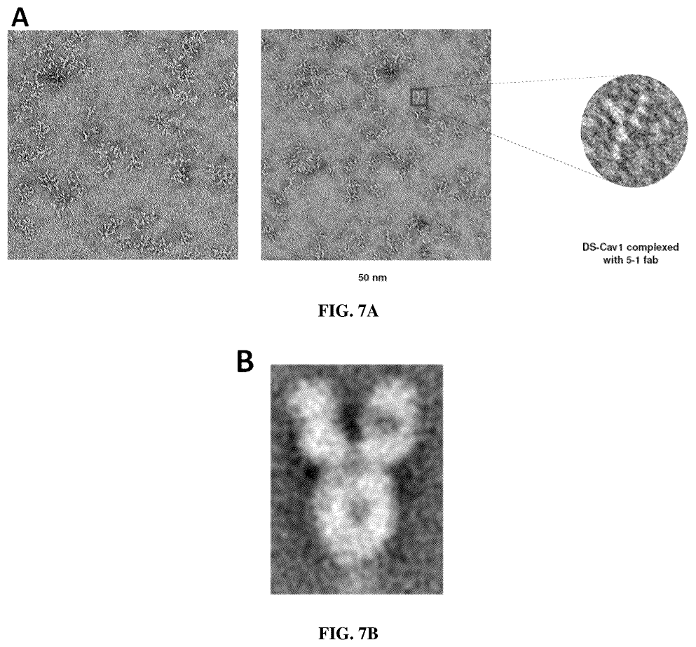

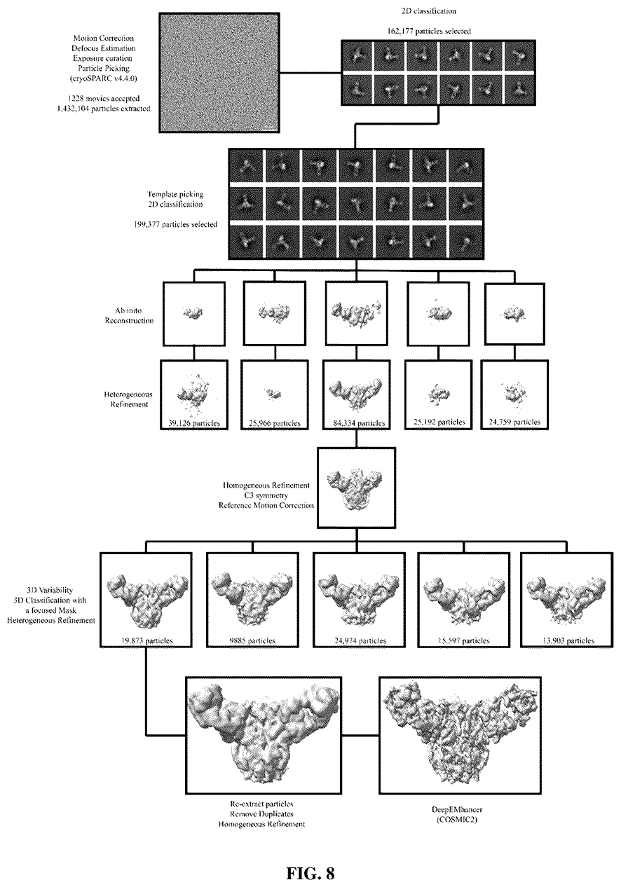

As mAb 5-1 was the most potently neutralizing antibody and displayed a unique competition profile that was not resolved by competition biolayer interferometry, the epitope of mAb 5-1 was investigated using negative stain electron microscopy (EM) and cryo-electron microscopy (cryoEM). Efforts with a prefusion RSV F protein (DS-Cav1) and 5-1 antigen-binding fragment (Fab) were unsuccessful, as most of the trimers were observed in a splayed-open state (FIGS. 7A and 7B). Therefore, a prefusion-stabilized hMPV F construct (hMPV F-DS-CavEs2-IPDS) was used, which contains intra- and inter-protomer disulfide bonds to lock hMPV F in a closed prefusion trimer conformation.

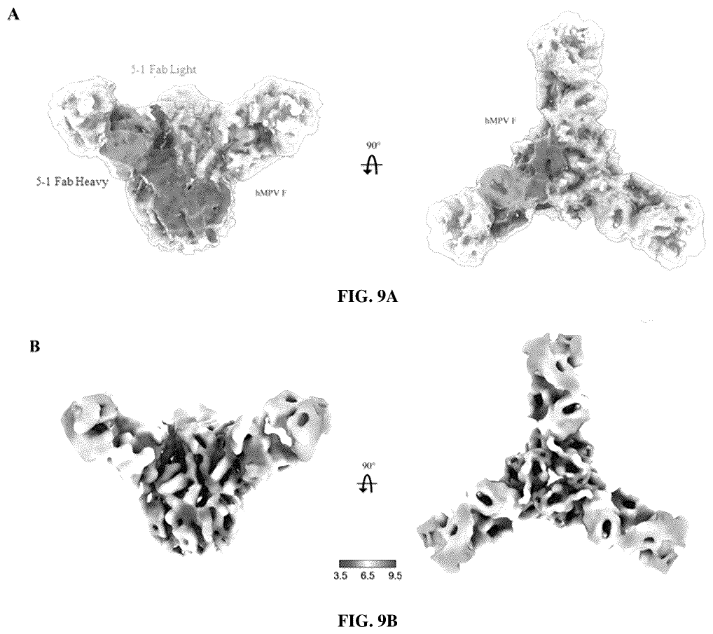

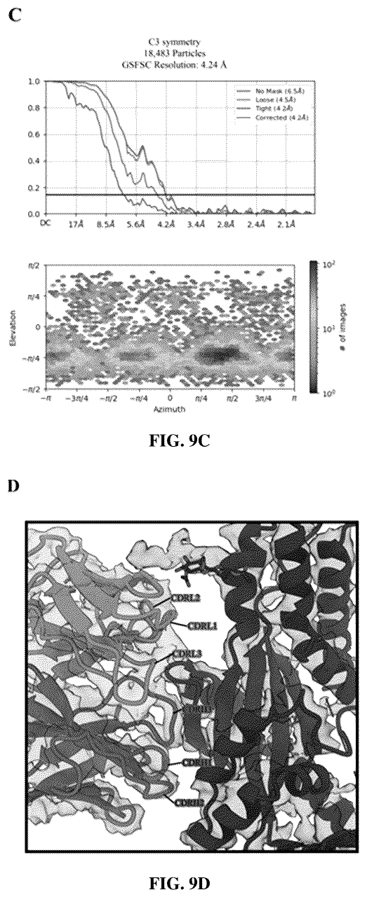

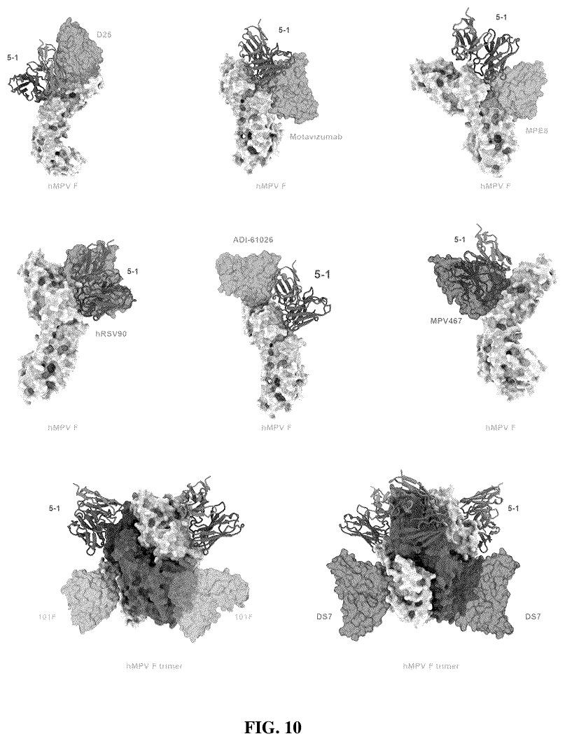

Cryo-EM analysis of hMPV F and 5-1 Fab revealed a heterogeneous mixture of complexes composed of three Fabs per trimer, with the majority of the particles displaying flexibility at the membrane-proximal base of the F protein (FIG. 9). However, a subset of particles retained after 2D classification were identified with a well-ordered base (˜23%), and further processing yielded a 3D reconstruction with a global resolution of 4.3 Å (FIGS. 10B and 10C). The cryo-EM map agrees very well with a model of the complex produced with AlphaFold3, and only light refinement was required to obtain an excellent map-to-model fit.

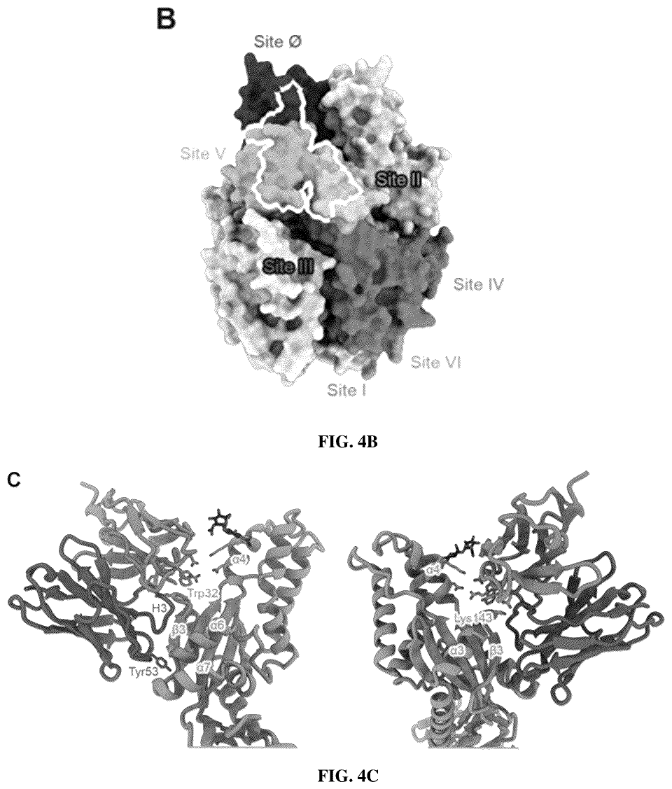

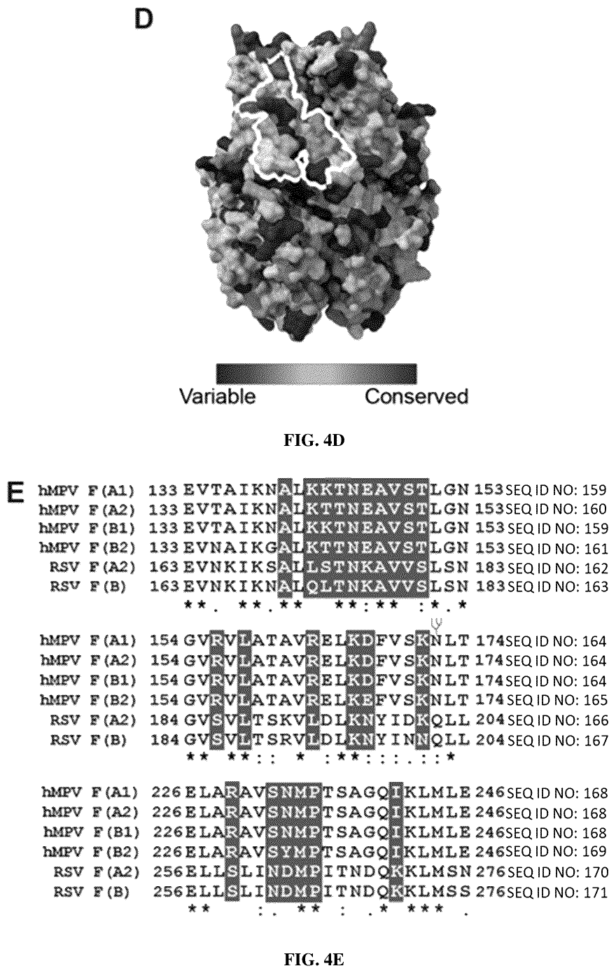

The structure reveals that the 5-1 epitope is contained within the F1 subunit of a single protomer and primarily spans antigenic sites II and V, with some additional interactions with site Ø (FIGS. 4A and 4B). The 5-1 heavy and light chains bury 597 Å2 and 303 Å2 of surface area, respectively, with the complementarity-determining region (CDR) 1 and 2 of the light chain contributing to the interaction with site Ø and the top half of site V. The light chain primarily interacts with residues on α4 through an electrostatic interaction network formed by Asp31_{CDRL1} and Arg50_{CDRL2} with RSV Fresidues Lys171 and Asp167, and with residues on the loop preceding β3 through the electrostatic interaction of Glu55_{CDRL2} with Lys143 (FIG. 4C). The heavy chain packs its CDR loops against the cleft between β3 and α6, with Tyr53_{CDRH2} inserted into the cleft. Interestingly, the 5-1 CDRL3 only interacts with residues on the CDRH2 and CDRH3 loops rather than with hMPV F, which may be important for stabilizing the heavy chain interactions (FIG. 4C). In addition, there appear to be interactions between light chain framework residues and the N-linked glycans attached to Asn172 on hMPV F, despite the low resolution and partially modeled glycan chains (FIGS. 10A, 10B, 10C, and 10D).

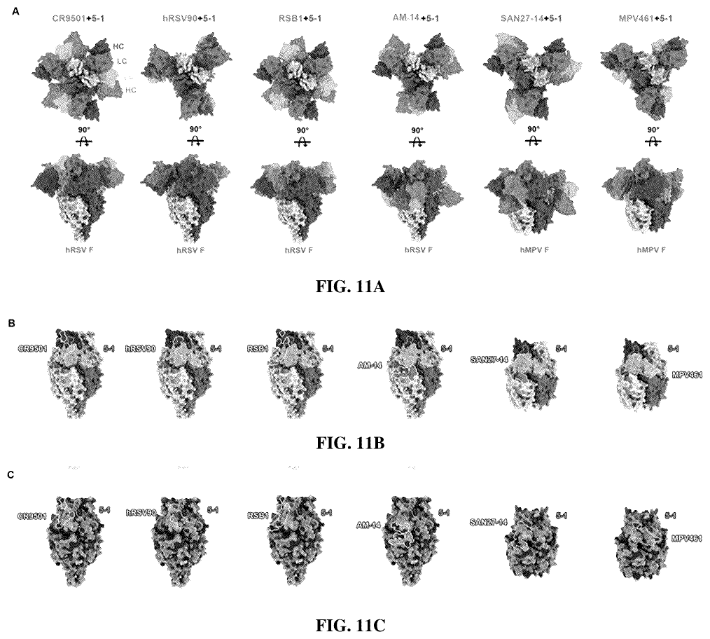

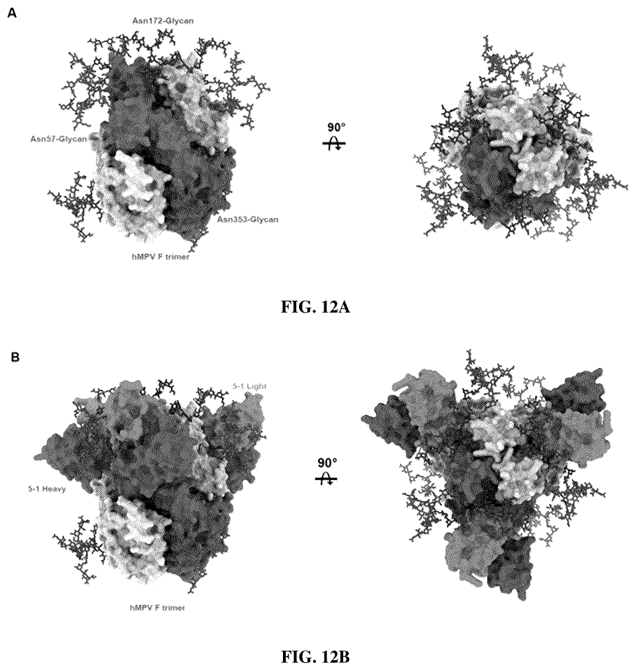

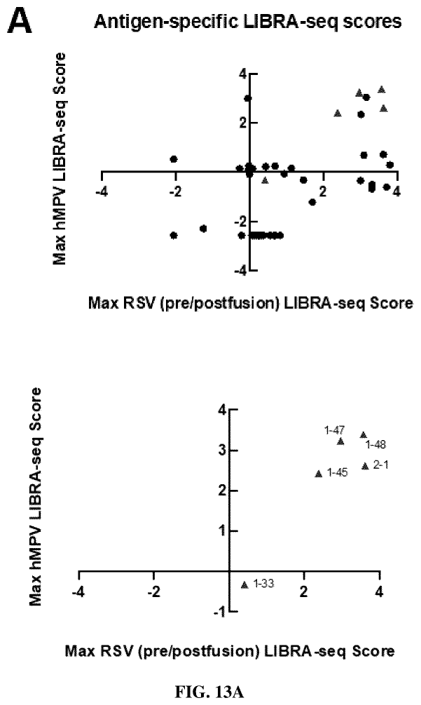

The structural model obtained from cryo-EM analysis agrees well with the ELISA and BLI competition binding data. Superposition of the cryo-EM structure with previously determined structures of the antibodies used in the competition assays predicts that 5-1 would sterically clash with D25, motavizumab, MPE8, hRSV90, ADI-61026 and MPV467 (FIG. 11). Further comparison to known hMPV and RSV F antibody complexes revealed that hRSV90 binds to a similar epitope on RSV F, except with an inverted arrangement of the heavy and light chains (FIG. 12). However, hRSV90 is specific for RSV and does not bind or neutralize hMPV.

The 5-1 epitope contains some amino acids that are not well conserved among RSV and hMPV F proteins, yet the antibody binding mode can accommodate these differences (FIGS. 4D and 4E). The substitutions will likely impact the affinity of 5-1 to different extents, but they do not introduce clashes that would prevent antibody binding. The region including the β3 strand is generally well conserved (hMPV F residues 142-150), as is the cleft between β3 and α6, into which Tyr53_{CDRH2} inserts. Thus, the structure and AlphaFold3 models of 5-1 bound to hMPV F and RSV F provide a structural basis for how 5-1 can bind an epitope at the F apex that is thought to be under immune pressure and less conserved than other regions.

### In-Vivo Protection Against Viral Infection

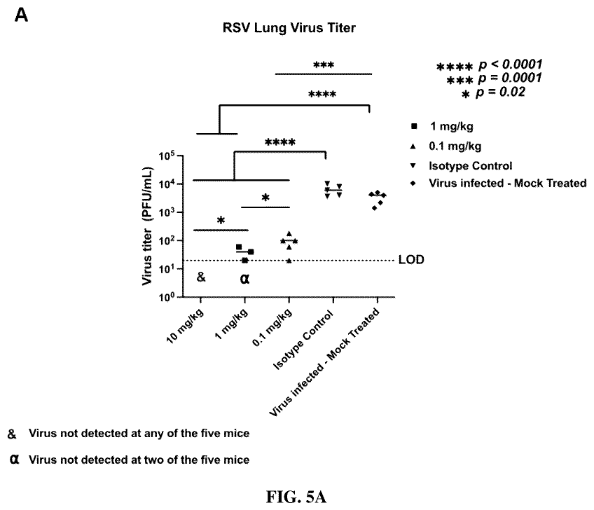

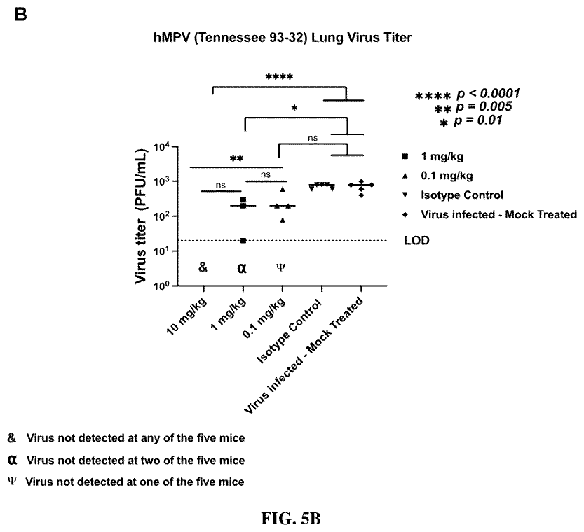

Next, the protective efficacy of mAb 5-1 in both an RSV and hMPV infection model was investigated in BALB/c mice. Fourteen-week-old female mice were mock treated with PBS, an isotype control human mAb VRC01, or different doses of mAb 5-1 six hours prior to intranasal RSV or hMPV challenge (FIGS. 5A and 5B). Lung viral titers of mice were determined by plaque assay on day 6 post infection to assess mAb 5-1 prophylaxis against infection. At the highest mAb 5-1 dose of 10 mg/kg, viral lung titers were below the detection limits for both RSV and hMPV for all animals (FIG. 5B). Even at the 10-fold lower dose of 1 mg/kg, 2/5 animals (40%) showed no detectable viral titers in the lung for both RSV and hMPV and were overall significantly lower than those observed in the control groups. Animals receiving the lowest dose of 0.1 mg/kg of mAb 5-1 showed significantly reduced lung viral titers for RSV and a 3.33-fold (though not statistically significant) reduction for hMPV. Together, these results showcase the in vivo protective ability of mAb 5-1 against RSV and hMPV challenge.

### DISCUSSION

Respiratory illness associated with infection by either RSV and/or hMPV remains a public health threat, with the potential for severe disease in neonatal, geriatric, and immunocompromised patients such as those undergoing hematopoietic stem cell transplant and patients suffering from pulmonary co-morbidities. While strategies to prevent severe infection induced by RSV have advanced in the last year, there are currently no approved treatments for infection by hMPV. Those with skill in the art have isolated RSV and hMPV cross-neutralizing antibodies that present an interesting alternative to mono-valent therapies, providing a protective regimen for the prevention or amelioration of disease caused by either mono- or co-infection of RSV and hMPV.

Five antibodies, targeting three previously reported epitopes on the F protein known to elicit cross-reactive humoral responses, were discovered. Consistent with the enrichment of site III-directed antibodies encoded by IGHV3-11/3-21: IGLV1-40, mAbs 9-1 and 2-6 display competition profiles indicative of binding at antigenic site III. Interestingly, germline-reverted mAbs 9-1 and 2-6 favored binding to F from different viruses, despite targeting the same site. Loss of antigenic binding of mAb 0-20 to both RSV and hMPV F in the germline state suggests cross-reactivity can be achieved through multiple antibody evolution pathways, i.e., through subsequent activation of either RSV or hMPV-specific B cells.

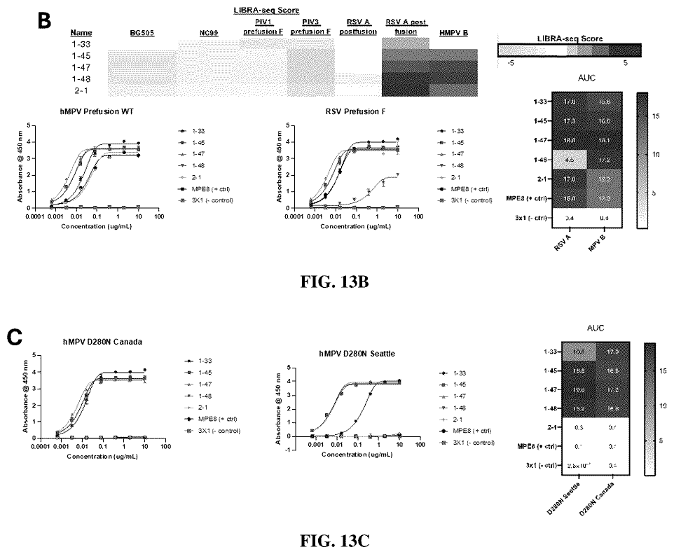

All five RSV/hMPV antibodies displayed in vitro neutralizing activity against infection by at least one representative virus of each genotype, albeit some mAbs displayed preferential neutralization against RSV or hMPV alone. mAb 5-1 displayed potent neutralization against all viruses tested, reaching neutralization potencies of better than 10 ng/mL IC₅₀ against hMPV 97-83 and hMPV TN/93-32. A significant proportion of hMPV field strains contain amino acid substitution D280N, which may impede binding of IGHV3-11/3-21: IGLV1-40 site III cross-reactive antibodies such as MPE8, 25P13, and RSV199. The structural analysis demonstrates this mutation would be well tolerated, as D280 falls outside of the epitope of 5-1, which is predominantly within antigenic site V and antigenic site II, with additional contacts with site Ø. The structure agrees well with the ELISA and BLI competition assay data, with the exception for antibody DS7. The modeling indicates that DS7 is not predicted to clash with 5-1, however some competition was observed (FIG. 2B). This may be influenced by the ability of DS7 to bind a conformation of the hMPV F protomer that contains elements of both the prefusion and postfusion conformation. The apex of hMPV F is shielded by glycans on Asn57 and Asn172 (FIG. 13 reducing antigenic exposure and dampening the immune response, relative to that of RSV, against site V and site Ø. However, despite this immune evasion technique, the human immune system has proven its ability to circumvent this obstacle through penetration of the glycan shield, as demonstrated with antibody ADI-61026, where ADI-61026 positions itself into a pocket between two glycans and directly interacts with Asn57-glycan. Glycan-shield-penetrating antibodies have also been reported that bind to HIV-1 Env, and hepatitis C E2. Herein it is demonstrated that 5-1 is also able to breach the glycan shield at the apex of hMPV F (FIG. 13).

As mAb 5-1 predominantly targets antigenic site V and provides protection against hMPV and RSV infection, the 5-1 binding pose and epitope was systematically compared with other site V antibodies, where structural information was available. Antibodies that bind to site V with varied modes of binding and thus contact differential residues in their respective epitopes, as observed with site III and IV binders. While many of the antibodies discussed here engage site V contact residues that are conserved between hMPV and RSV, the majority of these antibodies retain specificity for RSV or hMPV alone, likely as a result of the structural difference between RSV and hMPV trimers (FIG. 12).

Structural and repertoire analyses, in the context of antibodies elicited as a result of natural infection by RSV and hMPV, have revealed the propensity of site V towards the induction of potently neutralizing humoral responses. Within the trimeric prefusion F protein, the fusion peptide is buried inside a hydrophobic cavity occluded by the site V epitope. As demonstrated with a previously reported antibody targeting site V on hMPV F, one explanation for the potency of mAb 5-1, as compared to the other mAbs in the set, is that binding of mAb 5-1 prevents extension of the fusion peptide from the F protein, thereby disrupting the conformational changes necessary for productive infection.

Currently, no FDA-approved prophylaxis or therapeutics against hMPV F are available, despite substantial efforts. Recent progress, including structure-based RSV vaccines and antibody prophylaxis have been made, yet an antibody that potently neutralizes RSV with a unique antigenic footprint may offer additional benefits when considering virus evolution. Furthermore, an antibody that provides cross protection against both RSV and hMPV infection can be utilized to provide long-lasting protection against infection from either of these viruses in at-risk populations, providing important logistical advantages over developing multiple virus-specific mAbs. mAb 5-1 therefore presents an attractive target for further translational development.

### Materials and Methods

### Data Mining

LIBRA-seq datasets generated from 2020-2023 that included prefusion RSV A F, RSV B F, hMPV A F, and hMPV B F in the antigen screening library were mined for B cells displaying a minimum LIBRA-seq score of one for at least one of the F antigens, while also displaying a score below one for a control antigen, in this case, recombinant HIV-1 envelope protein. LIBRA-seq experiments were performed on peripheral blood mononuclear cells (PBMCs) samples obtained from otherwise healthy adult individuals. The established LIBRA-seq pipeline was used for score generation.

### Antibody Expression and Purification

For each antibody, variable genes were synthesized as cDNA and were inserted into bi-cistronic plasmids encoding for the constant regions of the heavy chain and either the kappa or lambda light chain (Twist BioScience). Antibodies were transiently expressed with Expifectamine transfection reagent (Thermo Fisher Scientific) in Expi293F cells in FreeStyle F17 expression media (Thermo Fisher) (0.1% Pluronic Acid F-68 and 20% 4 mM L-glutamine). Cells were cultured for 5 days at 8% CO₂ saturation and 37° C. with shaking. Five days post transfection, cells were collected and centrifuged at a minimum of 6000 rpm for 20 minutes. Supernatant was filtered with Nalgene Rapid Flow Disposable Filter Units with PES membrane (0.45 or 0.22 μm) and purified over protein A equilibrated with PBS. Antibodies were eluted with 100 mM glycine HCl at pH 2.7 directly into a 1:10 volume of 1 M Tris-HCl pH 8 and then exchanged into PBS for storage at 4° C.

### Enzyme Linked Immunosorbent Assay (ELISA)

Recombinant antigen was plated at 2 μg/mL overnight at 4° C. The next day, plates were washed three times with PBS supplemented with 0.05% Tween20 (PBS-T) and coated with 1% bovine serum albumin (BSA) in PBS-T. Plates were incubated for one hour at room temperature and then washed three times with PBS-T. Primary antibodies were diluted in 1% BSA in PBS-T, starting at 10 μg/mL with a serial 1:5 dilution, plated, and then incubated at room temperature for one hour before washing three times in PBS-T. The secondary antibody, goat anti-human IgG conjugated to peroxidase, was added at 1:10,000 dilution in 1% BSA in PBS-T to the plates, which were incubated for one hour at room temperature. Plates were washed three times with PBS-T and then developed by adding TMB substrate to each well. The plates were incubated at room temperature for five minutes, and then 1 N sulfuric acid was added to stop the reaction. Plates were read at 450 nm. ELISAs were performed in technical and biological duplicate.

Competitive Binding of mAbs with Site-Specific Antibodies in the Literature

Wells of 384-well microtiter plates were coated with 25 μl of 2 μg/mL purified F antigenic protein at 4° C. overnight. Plates were blocked with 50 μl of 1% BSA in PBS-T for 1 h before washing three times with PBS-T. Primary antibodies at 10 μg/mL were added to wells (20 μL per well) in duplicate and incubated for 1 h at room temperature. A biotinylated preparation of recombinantly produced site-specific monoclonal antibodies were added to wells of each primary antibody at a concentration of 10 μg/mL in a volume of 5 μL per well, without washing of unlabeled antibody, and then incubated for 1 h at room temperature. Plates were washed three times with PBS-T and bound antibodies were detected using horseradish peroxidase (HRP)-conjugated anti-biotin 1:1000 (ThermoFischer Scientific) and a TMB substrate. The signal obtained for binding of the biotin-labelled reference antibody in the presence of the unlabeled tested antibody was expressed as a percentage of the binding of the reference antibody alone after subtracting the background signal. Tested mAbs were considered competing if their presence reduced the reference antibody binding to less than 40% of its maximal binding and non-competing if the signal was greater than 71%. A level of 41 to 70% was considered intermediate competition.

### Germline Reversion of BCRs

Nucleotide sequences for the heavy and light chains of the described antibodies were annotated using IMGT V-Quest. Mutations occurring outside of the CDR3 region were reverted to the residues present in the V and J genes and alleles that most closely aligned to the mature sequence.

### Cell Culture and Virus CPE Determination

LLC-MK2 cells were obtained from ATCC (CCL-7) and grown in growth media (Opti-MEM with 2% FBS) at 37° C., 5% CO₂. Propagated virus was grown in viral growth media (Opti-MEM with 5 μg/mL trypsin-EDTA and 1% antibiotic-antimycotic) in LLC-MK2 cells at a multiplicity of infection (MOI) of 0.01 for 3-5 days at 37° C., 5% CO₂ until CPE was observed. Virus was harvested using the freeze-thaw method into 25% sucrose solution and stored at −80° C. until use.

Plaque Reduction Neutralization Test with MPV (CAN/97-83 and TN/93-32) or RSV (A2 and B) Virus

24 hours prior to viral infection, LLC-MK2 (for hMPV) or HEp-2 (for RSV) cells were plated in growth media at 5×10⁴ cells per well in 24 well plates and incubated at 37° C., 5% CO₂. The day of viral infection, mAbs were serially diluted in Opti-MEM with a starting concentration of 40 μg/mL. hMPV (CAN/97-83 and TN/93-32) or RSV (A2 and B) virus was diluted in Opti-MEM to a final concentration of 2400 plaque forming units (pfu)/mL and added to the mAb mixtures at a 1:1 volume ratio. The mAb/virus mixture incubated for 1 hour at room temperature. Prior to adding the mAb/virus mixture to cells, confluent cells in 24 well plates were washed gently three times with PBS. mAb/virus mixture was added to each well (50 μL per well) and the plates rocked at 37° C., 5% CO₂ for 1 hour. Warm overlay (0.75% methylcellulose in Opti-MEM, 5 μg/mL trypsin-EDTA and 1% antibiotic-antimycotic) was added to each well and the plates incubated for 4 days at 37° C., 5% CO₂. Following incubation, the cells were fixed with 10% neutral buffered formalin, washed with water three times, then blocked with milk blocking buffer (2% milk powder, 2% goat serum in PBS-T). Plates were washed three times with water and immunostained with human mAbs MPV364 (for hMPV) or 101F (for RSV) diluted to 5 μg/mL in milk blocking solution for 1 hour at room temperature. Plates were washed three times with water before adding the secondary antibody, goat anti-human IgG Fc conjugated to horse radish peroxidase, at a dilution of 1:2000 in milk blocking solution and incubated for 1 hour at room temperature. Plates were washed three times with water and developed with TrueBlue substrate by rocking for 10 minutes. After plaques were visibly stained by the substrate, the plates were washed once with water to stop the developing reaction. Immunostained plaques were counted and graphed on GraphPad Prism9.

RSV and hMPV Mouse Challenge Model

BALB/c mice (14 weeks old; The Jackson Laboratory) were intranasally infected with RSV A2 (2.0E+6 PFU/mouse) or hMPV TN/93-32 (3.0E+5 PFU/mouse) and euthanized 6 d postinfection. Monoclonal antibody 5-1 was administered intraperitoneally at 10, 1.0, or 0.1 mg/kg. Control mice were intraperitoneally injected with PBS or VRC01 (isotype control) at 10 mg/kg. All injections occurred 6 h prior to infection. Lung homogenates were used for viral titration by plaque assay as described above.

### Hep-2 Cell Immunofluorescence Assay to Detect mAb Autoreactivity

HEp-2 cell coated slides (BION ENTERPRISES LTD ANA (Hep-2) Test System, ANK-120) were incubated with purified antibodies at 10 and 1 μg/ml or control sera in a moist chamber at room temperature for 30 min. Controls provided with the kit included anti-nuclear antibody (ANA)⁺ and (ANA)⁻ human sera. Slides were washed twice with PBS for 5 min. Cells were stained with FITC-goat anti-human Ig per the manufacturer's instructions and incubated in a moist chamber at room temperature for 30 min. Slides were washed twice with PBS for 5 min, mounted with DAPI mounting medium (Southern Biotech 0100-20) and visualized by fluorescence microscopy (Olympus BX60 epifluorescence microscope coupled with a CCD camera and MagnaFire software Optronics International) at 40× magnification. Image brightness and contrast were optimized using Adobe Photoshop.

### Recombinant Protein Production for Negative Stain and Cryo-EM

Prefusion RSV-F strain A2 (DS-Cav-1) was used for negative stain-EM. Prefusion hMPV-F construct DS-CavEs2-IPDS protein was used for cryo-EM structural studies. In brief, plasmids encoding antigens were transfected into FreeStyle 293F cells (ThemoFisher) by PEI. Kifunensine and Pluronic F-68 (Gibco) were introduced 3 h post transfection. Six days later, the cell supernatant was filtered, and buffer exchanged into PBS by tangential flow filtration. Then, Step-TactinXT 4 Flow resin (IBA) was used to purify the protein from the filtered supernatant following the manufacturer's instruction. The purified protein was then concentrated using a 10 kDa molecular weight cutoff Amicon Ultra-15 centrifugal filter unit (Millipore) and subject to a Superose 6 increase 10/300 column (Cytivia) in PBS running buffer (hMPV-F DS-CavEs2-IPDS) or 2 mM Tris pH 8.0, 200 mM NaCl, and 0.02% NaN₃ (RSV A2 DS-Cav-1) for preparative size-exclusion chromatography. Peaks corresponding to trimeric species were identified based on elution volume and SDS-PAGE of elution fractions. Fractions containing pure fusion protein were pooled.

### Negative Stain-EM

For screening and imaging of negatively stained 5-1 Fab in complex with RSV-F A2 DS-Cav-1, sample was diluted to 100 mg/mL with buffer containing 10 mM NaCl, 20 mM HEPES buffer, pH 7.4, and 5% glycerol and applied to glow-discharged grid with continuous carbon film on 400 square mesh copper EM grids (Electron Microscopy Sciences). The grids were stained with 2% uranyl formate (UF). Grids were examined on a 100 kV Morgagni microscope with a 1k×1k AMT CCD camera.

### Cryo-EM Sample Preparation and Data Collection.

The purified hMPV-F-DS-CavEs2-IPDS was combined with 5-1 Fab in PBS buffer with a final concentration of 4.8 μM and 21.6 μM and incubated on ice for 3 min. Then, the 3 μl mixture was applied to an UltrAuFoil R1.2/1.3 300 mesh grid (Electron Microscopy Sciences) that had been glow-discharged with a PELCO easiGlow glow discharge cleaning system for 1 min. Grids were plunge-frozen using a Vitrobot Mark IV (ThermoFisher Scientific) at 4° C., 100% humidity. Blot settings were 4 s of blotting with force 2. Movies (3,538) were collected from a single grid on a 200 kV Glacios microscope (ThermoFisher Scientific) equipped with a Falcon 4 direct electron detector (ThermoFisher Scientific). Data were collected at a 50-degree tilt and at a magnification of 150,000×, where the calibrated pixel size is 0.94 Å/pix and the total exposure is 48.6 e⁻/Å².

### Cryo-EM Data Processing

Movies were imported into cryoSPARC v4.4.0 for gain correction, motion correction, patch CTF estimation, micrograph curation, particle picking, and particle extraction with a 2× Fourier crop. After two rounds of particle curation through 2D class averaging, the generated 2D class averages were used as templates to perform another round of template-based particle picking. Then, the particles were curated by 2D class averaging and curated particles were subject to ab initio reconstruction, heterogeneous refinement, and homogeneous refinement with C3 symmetry applied. Due to the presence of flexibility at the bottom region of the homogeneous-refined EM map, a 3D variability analysis job was performed with a focused mask to explore alternative conformations. After 3D variability analysis, a 3D classification job with a focused mask on the hMPV F base region was executed to generate EM maps of different conformations, followed by heterogeneous refinement. As particles were processed with Fourier cropping in the procedure described above, we re-extracted the particles with raw pixel size, removed the duplicate particles and reconstructed one EM map with homogeneous refinement and reference-based motion correction. Finally, the map from the last round of homogeneous refinement was sharpened using DeepEMhancer. For model building, an initial model was generated by AlphaFold3 server. As the predicted model aligned well with our 3D EM map, the following iterative refinements were performed using this model in Coot, PHENIX, and ISOLDE. The adjacent cystines in 5-1 Fab CDRH3 loop were modeled as a disulfide bond in the AlphaFold3 predicted model and were left unchanged during refinement. At the last round of refinement, glycans were built into the model, refined and validated using Coot and Privateer software. The EM processing workflow is shown as FIG. 9 and EM validation results are shown in FIG. 10. Refinement statistics are shown in Table 3.

### Sequence Conservation Analysis and Alignment

The glycoprotein sequence of hMPV F protein from strain NL/1/100 (A1 sub lineage, NCBI accession: YP_009513268.1) was uploaded into the HMMER web server to search for homologous sequences against UniProtKB database with phmmer programs and default parameters. The searching results were then manually filtered based on species, similarity, coverage and hit position. To avoid potential bias, 250 sequences for both hMPV F and RSV F were extracted from the search results and aligned with Clustal Omega. The output was imported into ChimeraX to generate a sequence conservation map. For direct alignment of four representative hMPV F and two RSV F protein sequences, hMPV F from A1 (NL/1/00 strain, NCBI accession: NC_039199.1), A2 (NL/17/00 strain, NCBI accession: AAQ90144.1), B1 (NL/1/99 strain, NCBI accession: AAQ90145.1), B2 (NL/1/94 strain, AAQ90146.1) and RSV F from A2 (NCBI accession: ACO83301.1) and B (NCBI accession: WKU63582.1) sequences were pooled and aligned with Clustal Omega.

### Data Availability

Datasets from which individual antibody sequences were pulled can be found in Abu-Shmais et al. (Abu-Shmais, A. A., Miller, R. J., Janke, A. K., Wolters, R. M., Holt, C. M., Raju, N., Carnahan, R. H., Crowe, J. E., Jr., Mousa, J. J., and Georgiev, I. S. (2024). Potent HPIV3-neutralizing IGHV5-51 Antibodies Identified from Multiple Individuals Show L Chain and CDRH3 Promiscuity. J Immunol 212, 1450-1456. 10.4049/jimmunol.2300880) and Abu-Shmais et al. (Abu-Shmais, A. A., Vukovich, M. J., Wasdin, P. T., Suresh, Y. P., Rush, S. A., Gillespie, R. A., Sankhala, R. S., Choe, M., Joyce, M. G., Kanekiyo, M., et al. (2023). Convergent Sequence Features of Antiviral B Cells. bioRxiv, 2023.2009.2006.556442. 10.1101/2023.09.06.556442). The EM map and coordinates for the hMPV F and 5-1 Fab complex have been deposited into the Electron Microscopy Data Bank (EMDB-45412) and the Protein Data Bank (9CB1; DOI: doi.org/10.2210/pdb9CB1/pdb). All data are included in the article and/or supporting information.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Sequence characteristics of RSV/hMPV cross-reactive antibodies. Percent identity is
calculated at the nucleotide level and sequences and VDJ/VJ length are displayed at
the amino acid level.

| | Native | VH | VH % | | SEQ ID | CDRH3 |
|---|---|---|---|---|---|---|
| mAb | Isotype | Gene | Identity | CDRH3 | NO | Length |
| 0-20 | IGHG1 | IGHV3- | 87.5 | ARGNNLF | 59 | 15 |
| | | 11 | | DDRGLFDH | | |
| 5-1 | IGHA1 | IGHV1- | 88.89 | ARGPCCSS | 63 | 14 |
| | | 18 | | PRPYDI | | |
| 2-6 | IGHG2 | IGHV3- | 93.4 | ARISYTSTG | 65 | 15 |
| | | 11 | | PFYFDS | | |
| 9-1 | IGHG1 | IGHV3- | 89.58 | ARDSGQQL | 67 | 13 |
| | | 21 | | DPFDY | | |
| 1-2 | IGHG3 | IGHV3- | 93.4 | ARAAYDSL | 158 | 13 |
| | | 30 | | TYFEF | | |
| | Native | VL | VL % | | SEQ ID | CDRL3 |
| mAb | Isotype | Gene | Identity | CDRL3 | NO | Length |
| 0-20 | IGHG1 | IGLV3- | 89.47 | QVRDTG | 60 | 11 |
| | | 21 | | TFQHV | | |
| 5-1 | IGHA1 | IGKV1-5 | 94.2 | QQCYTY | 64 | 9 |
| | | | | SQT | | |
| 2-6 | IGHG2 | IGLV1- | 97.22 | QSYDRSL | 66 | 11 |
| | | 40 | | SGYV | | |
| 9-1 | IGHG1 | IGLV1- | 94.79 | QSYDKRL | 68 | 11 |
| | | 40 | | FGWV | | |
| 1-2 | IGHG3 | IGLV3- | 94.98 | QVWDSTS | 62 | 11 |
| | | 21 | | DHWV | | |

TABLE 2
The LIBRA-seq scores (LSSs) and sequence characteristics for each cross-reactive
antibody (rows) shown are the CDR amino acid sequences and lengths, V-gene and
J-gene and percentage of nucleotide identities (columns).

| | Native | VH | JH | VH | JH | | SEQ ID | CDRH3 |
|---|---|---|---|---|---|---|---|---|
| mAb | Isotype | Gene | Gene | Identity | Identity | CDRH3 | NO | Length |
| 1-33 | IGHG2 | IGHV3- | IGHJ6 | 0.92 | 0.86 | CARGGRL | 148 | 22 |
| | | 7 | | | | RHFGNYY | | |
| | | | | | | YYPGMGV | | |
| | | | | | | W | | |
| 1-45 | IGHG1 | IGHV3- | IGHJ3 | 0.84 | 0.82 | CARVHLG | 149 | 16 |
| | | 21 | | | | RQTHGFD | | |
| | | | | | | LW | | |
| 1-47 | IGHG2 | IGHV3- | IGHJ3 | 0.94 | 0.92 | CARVHLG | 150 | 16 |
| | | 21 | | | | RQTYGFDI | | |
| | | | | | | W | | |
| 1-48 | IGHG1 | IGHV3- | IGHJ4 | 0.89 | 0.90 | CARDGFG | 151 | 17 |
| | | 23 | | | | DSSGQELD | | |
| | | | | | | YW | | |
| 2-1 | IGHG1 | IGHV3- | IGHJ5 | 0.90 | 0.96 | CVRSLYDS | 152 | 20 |
| | | 21 | | | | GHYYNPK | | |
| | | | | | | WFDPW | | |
| | Native | VL | JL | VL | JL | | SEQ ID | CDRL3 |
| mAb | Isotype | Gene | Gene | Identity | Identity | CDRL3 | NO | Length |
| 1-33 | IGHG2 | IGKV2- | IGHJ6 | 0.09 | 0.97 | CMQSLQTPLTF | 153 | 11 |
| | | 28 | | | | | | |
| 1-45 | IGHG1 | IGLV1- | IGHJ3 | 0.93 | 0.89 | CQSFDRSLDGYAF | 154 | 13 |
| | | 40 | | | | | | |
| 1-47 | IGHG2 | IGLV1- | IGHJ3 | 0.95 | 0.97 | CHSYDRSLGGYVF | 155 | 13 |
| | | 40 | | | | | | |
| 1-48 | IGHG1 | IGKV2- | IGHJ4 | 0.95 | 1.00 | CMQALQTRTF | 156 | 10 |
| | | 28 | | | | | | |
| 2-1 | IGHG1 | IGLV1- | IGHJ2 | 0.941 | 0.89 | CHSYDRTLREVF | 157 | 12 |
| | | 40 | | | | | | |

TABLE 3
Cryo-EM data collection and reconstruction statistics.

| EM data collection | |
|---|---|
| EMDB | 45412 |
| Microscope | Glacios |
| Voltage (kV) | 200 |
| Detector | Falcon 4 |
| Magnification (nominal) | 150,000 |
| Pixel size (Å/pixl) | 0.94 |
| Exposure rate (e-/pixel/sec) | 3.26 |
| Exposure (e-/Å²) | 48.64 |
| Defocus range (μm) | 1.0-2.5 |
| Tilt angle (°) | 50 |
| Movies collected | 3,538 |
| Movies used | 1,228 |
| Particles extracted (total) | 1,432,104 |
| Automation software | SerialEM |
| Sample | hMPV F + 5-1 Fab |
| 3D reconstruction statistics | |
| Particles | 19,873 |
| Symmetry | C3 |
| Map sharpenning B factor | −140.1 |
| Umasked resolution at 0.5 FSC (Å) | 8.6 |
| Masked resolution at 0.5 FSC (Å) | 7.0 |
| Umasked resolution at 0.143 FSC (Å) | 6.5 |
| Masked resolution at 0.143 FSC (Å) | 4.2 |
| Model refinement and validation | |
| statistics | |
| PDB ID | 9CB1 |
| Refinement package | Phenix, Isolde, Coot, |
| | CCP4, Privateer |
| Refinement tools | Real space |
| | refinement |
| Refinement strategies | min global, adp |
| Composition | |
| Amino acids | 1968 |
| Glycan | 6 |
| RMSD bonds (Å) | 0.005 |
| RMSD angles (°) | 0.86 |
| Average B factors | 172.4 |
| Amino acids | |
| Ramachandran | |
| Favored (%) | 96.49 |
| Allowed (%) | 3.36 |
| Outliers (%) | 0.16 |
| Rotamer outliers (%) | 0.78 |
| Clash score | 6.8 |
| C-beta outliers (%) | NA |
| CaBLM outliers (%) | 2.58 |
| 0.5 FSC models (Å) | 4.4 |
| CC (mask) | 0.70 |
| MolProbity score | 1.61 |
| EMRinger score | 1.30 |

TABLE 4
Antibody Heavy Chain and Light Chain Complementarity Determining Regions (CDRs)

| | | | | | SEQ |
|---|---|---|---|---|---|
| Antibody | | | SEQ | | ID |
| Name | CDR | Heavy Chain | ID NO | Light Chain | NO: |
| 0-20 | 1 | GFKFNDYY | 45 | NIGSKS | 46 |
| | 2 | ISSFGVTT | 54 | DNT | — |
| | 3 | ARGNNLFDDRGLFDH | 59 | QVRDTGTFQHV | 60 |
| 1-2 | 1 | GFSFSNFG | 47 | NIGGKG | 48 |
| | 2 | ISHDGSNK | 55 | LDR | — |
| | 3 | DTAVYYCARAAYDSLTYFEF | 61 | QVWDSTSDHWV | 62 |
| 5-1 | 1 | AYPFGNYG | 49 | QSIDDW | 50 |
| | 2 | ISAYTGHT | 56 | RAS | — |
| | 3 | ARGPCCSSPRPYDI | 63 | QQCYTYSQT | 64 |
| 2-6 | 1 | GFTLSGYY | 51 | SSNIGAGYD | 52 |
| | 2 | VSGSSSYT | 57 | GNN | — |
| | 3 | ARISYTSTGPFYFDS | 65 | QSYDRSLSGYV | 66 |
| 9-1 | 1 | GFSITSFS | 53 | SSNIGAGYD | 52 |
| | 2 | ISASSSYI | 58 | GNN | — |
| | 3 | ARDSGQQLDPFDY | 67 | QSYDKRLFGWV | 68 |

TABLE 5
Antibody Heavy Chain and Light Chain Complementarity Determining Regions (CDRs)

| | | | | | SEQ |
|---|---|---|---|---|---|
| Antibody | | | SEQ | | ID |
| Name | CDR | Heavy Chain | ID NO | Light Chain | NO: |
| 1-33 | 1 | GFNFSDYR | 79 | QSLLHNNTYNY | 82 |
| | 2 | IKEDGREK | 80 | LAS | 83 |
| | 3 | ARGGRLRHFGNYYYYPG | 81 | MQSLQTPLT | 84 |
| | | MGV | | | |
| 1-45 | 1 | GFTISGYN | 95 | DSNIGAGYE | 98 |
| | 2 | ITGLGNYI | 96 | GYN | 99 |
| | 3 | ARVHLGRQTHGFDL | 97 | QSFDRSLDGYA | 100 |
| 1-47 | 1 | GFTFSGYN | 111 | SSNIGAGYD | 114 |
| | 2 | ITSGSNYI | 112 | GNI | 115 |
| | 3 | ARVHLGRQTYGFDI | 113 | HSYDRSLGGYV | 116 |
| 1-48 | 1 | GFTFSNYA | 127 | QSLLQSNGYNY | 130 |
| | 2 | VGASGYPT | 128 | LGF | 131 |
| | 3 | ARDGFGDSSGQELDY | 129 | MQALQTRT | 132 |
| 2-1 | 1 | GFRLSSYG | 143 | TSNIGAGYD | 146 |
| | 2 | ISGGSNYI | 144 | GNI | — |
| | 3 | VRSLYDSGHYYNPKWFDP | 145 | HSYDRTLREV | 147 |

What is claimed is:

1. A recombinant antibody comprising a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, or SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, or SEQ ID NO: 136.

2. The recombinant antibody of claim 1, wherein the heavy chain comprises:
a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143;
a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and
a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, or SEQ ID NO: 158.

3. The recombinant antibody of claim 1, wherein the light chain comprises:
a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146;
a CDR2 comprising amino acid sequences DNT, LDR, RAS, GNN, SEQ ID NO: 83, SEQ ID NO: 99, SEQ ID NO: 115, or SEQ ID NO: 131; and
a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, or SEQ ID NO: 157.

4. The recombinant antibody of claim 1, further comprising a heavy chain CDR1, CDR2, and CDR3 and a light chain CDR1, CDR2, and CDR3 selected from the group consisting of:
SEQ ID NO: 45, SEQ ID NO: 54, SEQ ID NO: 59, SEQ ID NO: 46, SEQ ID NO: 60, and amino acid sequence DNT;
SEQ ID NO: 47, SEQ ID NO: 55, SEQ ID NO: 61, SEQ ID NO: 48, SEQ ID NO: 62, and amino acid sequence LDR;
SEQ ID NO: 49, SEQ ID NO: 56, SEQ ID NO: 63, SEQ ID NO: 50, SEQ ID NO: 64, and amino acid sequence RAS;
SEQ ID NO: 51, SEQ ID NO: 57, SEQ ID NO: 65, SEQ ID NO: 52, SEQ ID NO: 66, and amino acid sequence GNN;
SEQ ID NO: 53, SEQ ID NO: 58, SEQ ID NO: 67, SEQ ID NO: 52, SEQ ID NO: 68, and amino acid sequence GNN;
SEQ ID NO: 79, SEQ ID NO: 80, SEQ ID NO: 81, SEQ ID NO: 82, SEQ ID NO: 83, and SEQ ID NO: 84;
SEQ ID NO: 95, SEQ ID NO: 96, SEQ ID NO: 97, SEQ ID NO: 98, SEQ ID NO: 99, and SEQ ID NO: 100;
SEQ ID NO: 111, SEQ ID NO: 112, SEQ ID NO: 113, SEQ ID NO: 114, SEQ ID NO: 115, and SEQ ID NO: 116;
SEQ ID NO: 127, SEQ ID NO: 128, SEQ ID NO: 129, SEQ ID NO: 130, SEQ ID NO: 131, and SEQ ID NO: 132; and
SEQ ID NO: 143, SEQ ID NO: 144, SEQ ID NO: 145, SEQ ID NO: 146, SEQ ID NO: 147, and SEQ ID NO: 115.

5. The recombinant antibody of claim 1, wherein the recombinant antibody comprises SEQ ID NO: 45, SEQ ID NO: 54, SEQ ID NO: 59, SEQ ID NO: 46, SEQ ID NO: 60, and amino acid sequence DNT.

6. The recombinant antibody of claim 1, wherein the recombinant antibody comprises SEQ ID NO: 47, SEQ ID NO: 55, SEQ ID NO: 61, SEQ ID NO: 48, SEQ ID NO: 62, and amino acid sequence LDR.

7. The recombinant antibody of claim 1, wherein the recombinant antibody comprises SEQ ID NO: 49, SEQ ID NO: 56, SEQ ID NO: 63, SEQ ID NO: 50, SEQ ID NO: 64, and amino acid sequence RAS.

8. The recombinant antibody of claim 1, wherein the recombinant antibody comprises SEQ ID NO: 51, SEQ ID NO: 57, SEQ ID NO: 65, SEQ ID NO: 52, SEQ ID NO: 66, and amino acid sequence GNN.

9. The recombinant antibody of claim 1, wherein the recombinant antibody comprises SEQ ID NO: 53, SEQ ID NO: 58, SEQ ID NO: 67, SEQ ID NO: 52, SEQ ID NO: 68, and amino acid sequence GNN.

10. The recombinant antibody of claim 1, wherein the recombinant antibody comprises SEQ ID NO: 79, SEQ ID NO: 80, SEQ ID NO: 81, SEQ ID NO: 82, SEQ ID NO: 83, and SEQ ID NO: 84.

11. The recombinant antibody of claim 1, wherein the recombinant antibody comprises SEQ ID NO: 95, SEQ ID NO: 96, SEQ ID NO: 97, SEQ ID NO: 98, SEQ ID NO: 99, and SEQ ID NO: 100.

12. The recombinant antibody of claim 1, wherein the recombinant antibody comprises SEQ ID NO: 111, SEQ ID NO: 112, SEQ ID NO: 113, SEQ ID NO: 114, SEQ ID NO: 115, and SEQ ID NO: 116.

13. The recombinant antibody of claim 1, wherein the recombinant antibody comprises SEQ ID NO: 127, SEQ ID NO: 128, SEQ ID NO: 129, SEQ ID NO: 130, SEQ ID NO: 131, and SEQ ID NO: 132.

14. The recombinant antibody of claim 1, wherein the recombinant antibody comprises SEQ ID NO: 143, SEQ ID NO: 144, SEQ ID NO: 145, SEQ ID NO: 146, SEQ ID NO: 147, and SEQ ID NO: 115.

15. The recombinant antibody of claim 1, wherein the antibody comprises an antigen-binding site to human respiratory syncytial virus (RSV) and human metapneumovirus (hMPV).

16. The recombinant antibody of claim 1, wherein the antibody comprises an antigen-binding site to RSV or hMPV.

17. A nucleic acid sequence encoding the recombinant antibody of claim 1.

18. An expression vector comprising the nucleic acid of claim 17.

19. A cell comprising the nucleic acid of claim 17.

20. A method of treating a respiratory infection in a subject in need thereof, the method comprising administering to the subject a recombinant antibody composition, wherein the recombinant antibody composition comprises a heavy chain sequence selected from SEQ ID NO: 11, SEQ ID NO: 13, SEQ ID NO: 15, SEQ ID NO: 17, SEQ ID NO: 19, SEQ ID NO: 71, SEQ ID NO: 87, SEQ ID NO: 103, SEQ ID NO: 119, or SEQ ID NO: 135, and a light chain sequence selected from SEQ ID NO: 12, SEQ ID NO: 14, SEQ ID NO: 16, SEQ ID NO: 18, SEQ ID NO: 20, SEQ ID NO: 72, SEQ ID NO: 88, SEQ ID NO: 104, SEQ ID NO: 120, or SEQ ID NO: 136, wherein the heavy chain comprises:
a complementarity determining region (CDR) 1 comprising SEQ ID NO: 45, SEQ ID NO: 47, SEQ ID NO: 49, SEQ ID NO: 51, SEQ ID NO: 53, SEQ ID NO: 79, SEQ ID NO: 95, SEQ ID NO: 111, SEQ ID NO: 127, or SEQ ID NO: 143;
a CDR2 comprising SEQ ID NO: 54, SEQ ID NO: 55, SEQ ID NO: 56, SEQ ID NO: 57, SEQ ID NO: 58, SEQ ID NO: 80, SEQ ID NO: 96, SEQ ID NO: 112, SEQ ID NO: 128, or SEQ ID NO: 144; and
a CDR3 comprising SEQ ID NO: 59, SEQ ID NO: 61, SEQ ID NO: 63, SEQ ID NO: 65, SEQ ID NO: 67, SEQ ID NO: 81, SEQ ID NO: 97, SEQ ID NO: 113, SEQ ID NO: 129, SEQ ID NO: 145, SEQ ID NO: 148, SEQ ID NO: 149, SEQ ID NO: 150, SEQ ID NO: 151, SEQ ID NO: 152, or SEQ ID NO: 158; and wherein the light chain comprises:
a CDR1 comprising SEQ ID NO: 46, SEQ ID NO: 48, SEQ ID NO: 50, SEQ ID NO: 52, SEQ ID NO: 82, SEQ ID NO: 98, SEQ ID NO: 114, SEQ ID NO: 130, or SEQ ID NO: 146;
a CDR2 comprising amino acid sequences DNT, LDR, RAS, GNN, SEQ ID NO: 83, SEQ ID NO: 99, SEQ ID NO: 115, or SEQ ID NO: 131; and
a CDR3 comprising SEQ ID NO: 60, SEQ ID NO: 62, SEQ ID NO: 64, SEQ ID NO: 66, SEQ ID NO: 68, SEQ ID NO: 84, SEQ ID NO: 100, SEQ ID NO: 116, SEQ ID NO: 132, SEQ ID NO: 147, SEQ ID NO: 153, SEQ ID NO: 154, SEQ ID NO: 155, SEQ ID NO: 156, or SEQ ID NO: 157.
